SEQUENCE LISTING (46 sequences)

SEQ ID NO: 1 - RNA, 19 nt, Artificial Sequence
  siRNA for Aim2
gauagaguacuguauggua

SEQ ID NO: 2 - RNA, 19 nt, Artificial Sequence
  siRNA for Nlrp3 1
ucucaagucuaagcaccaa

SEQ ID NO: 3 - RNA, 19 nt, Artificial Sequence
  siRNA for Nlrp3 2
caucaaugcugcuucgaca

SEQ ID NO: 4 - RNA, 19 nt, Artificial Sequence
  siRNA for Nlrp3 3
cagugacaauacucuggga

SEQ ID NO: 5 - RNA, 19 nt, Artificial Sequence
  siRNA for Asc 1
gagcagcugcaaacgacua

SEQ ID NO: 6 - RNA, 19 nt, Artificial Sequence
  siRNA for Asc 2
gcuacuaucuggagucgua

SEQ ID NO: 7 - RNA, 19 nt, Artificial Sequence
  siRNA for Asc 3
ccuggaaccugaccugcaa

SEQ ID NO: 8 - DNA, 22 bp, Artificial Sequence
  Il-1 F_primer
gcaactgttcctgaactcaact

SEQ ID NO: 9 - DNA, 21 bp, Artificial Sequence
  Il-1 R_primer
atcttttggggtccgtcaact

SEQ ID NO: 10 - DNA, 23 bp, Artificial Sequence
  Il-6 F_primer
tagtccttcctaccccaatttcc

SEQ ID NO: 11 - DNA, 21 bp, Artificial Sequence
  Il-6 R_primer
ttggtccttagccactccttc

SEQ ID NO: 12 - DNA, 21 bp, Artificial Sequence
  Il-23 F_primer
atgctggattgcagagcagta

SEQ ID NO: 13 - DNA, 23 bp, Artificial Sequence
  Il-23 R_primer
acggggcacattatttttagtct

SEQ ID NO: 14 - DNA, 21 bp, Artificial Sequence
  Il-12 F_primer
tggtttgccatcgttttgctg

SEQ ID NO: 15 - DNA, 22 bp, Artificial Sequence
  Il-12 R_primer
acaggtgaggttcactgtttct

SEQ ID NO: 16 - DNA, 21 bp, Artificial Sequence
  Il-10 F_primer
gctcttactgactggcatgag

SEQ ID NO: 17 - DNA, 20 bp, Artificial Sequence
  Il-10 R_primer
cgcagctctaggagcatgtg

SEQ ID NO: 18 - DNA, 19 bp, Artificial Sequence
  Tgf- F_primer
ctcccgtggcttctagtgc

SEQ ID NO: 19 - DNA, 22 bp, Artificial Sequence
  Tgf- R_primer
gccttagtttggacaggatctg

SEQ ID NO: 20 - DNA, 19 bp, Artificial Sequence
  Tnf- F_primer
caggcggtgcctatgtctc

SEQ ID NO: 21 - DNA, 22 bp, Artificial Sequence
  Tnf- R_primer
cgatcaccccgaagttcagtag

SEQ ID NO: 22 - DNA, 19 bp, Artificial Sequence
  Ifn- F_primer
gccacggcacagtcattga

SEQ ID NO: 23 - DNA, 21 bp, Artificial Sequence
  Ifn- R_primer
tgctgatggcctgattgtctt

SEQ ID NO: 24 - DNA, 19 bp, Artificial Sequence
  Nlrp3 F_primer
tggatgggtttgctgggat

SEQ ID NO: 25 - DNA, 21 bp, Artificial Sequence
  Nlrp3 R_primer
ctgcgtgtagcgactgttgag

SEQ ID NO: 26 - DNA, 19 bp, Artificial Sequence
  Gapdh F_primer
tcaccaccatggagaaggc

SEQ ID NO: 27 - DNA, 20 bp, Artificial Sequence
  Gapdh R_primer
gctaagcagttggtggtgca

SEQ ID NO: 28 - DNA, 23 bp, Artificial Sequence
  Human IL-1 F_primer
atgatggcttattacagtggcaa

SEQ ID NO: 29 - DNA, 20 bp, Artificial Sequence
  Human IL-1 R_primer
gtcggagattcgtagctgga

SEQ ID NO: 30 - DNA, 23 bp, Artificial Sequence
  Human IL-6 F_primer
actcacctcttcagaacgaattg

SEQ ID NO: 31 - DNA, 23 bp, Artificial Sequence
  Human IL-6 R_primer
ccatctttggaaggttcaggttg

SEQ ID NO: 32 - DNA, 22 bp, Artificial Sequence
  Human TNF- F_primer
cctctctctaatcagccctctg

SEQ ID NO: 33 - DNA, 21 bp, Artificial Sequence
  Human TNF- R_primer
gaggacctgggagtagatgag

SEQ ID NO: 34 - DNA, 23 bp, Artificial Sequence
  Human IFN- F_primer
tcggtaactgacttgaatgtcca

SEQ ID NO: 35 - DNA, 21 bp, Artificial Sequence
  Human IFN- R_primer
tcgcttccctgttttagctgc

SEQ ID NO: 36 - DNA, 22 bp, Artificial Sequence
  Human GAPDH F_primer
acaactttggtatcgtggaagg

SEQ ID NO: 37 - DNA, 19 bp, Artificial Sequence
  Human GAPDH R_primer
gccatcacgccacagtttc

SEQ ID NO: 38 - DNA, 25 bp, Artificial Sequence
  sgRNA for AIM2 F_primer
caccggcaagatattatcggcacag

SEQ ID NO: 39 - DNA, 25 bp, Artificial Sequence
  sgRNA for AIM2 R_primer
aaacctgtgccgataatatcttgcc

SEQ ID NO: 40 - DNA, 25 bp, Artificial Sequence
  sgRNA for NLRP3 F_primer
caccgctgattagtgctgagtaccg

SEQ ID NO: 41 - DNA, 25 bp, Artificial Sequence
  sgRNA for NLRP3 R_primer
aaaccggtactcagcactaatcagc

SEQ ID NO: 42 - DNA, 25 bp, Artificial Sequence
  sgRNA for ASC F_primer
caccgcgagggtcacaaacgttgag

SEQ ID NO: 43 - DNA, 25 bp, Artificial Sequence
  sgRNA for ASC R_primer
aaacctcaacgtttgtgaccctcgc

SEQ ID NO: 44 - DNA, 25 bp, Artificial Sequence
  sgRNA for caspase-1 F_primer
caccgctttaaaccacaccacacca

SEQ ID NO: 45 - DNA, 25 bp, Artificial Sequence
  sgRNA for caspase-1 R_primer
aaactggtgtggtgtggtttaaagc

SEQ ID NO: 46 - protein, 772 aa, Staphylococcus aureus
MKALLLKTSVWLVLLFSAMGLWQVSSAAEQHTPMKAHAVTTIDKATTDRQLVLPTKEAAHQSGEEAATNVSASAQGTADDTNNKVTSNAPSNKPSTAVSTTVNETHDVDAQQASTQKPTQSATFKLSNAKTASLSPRMFAANAPQTTTHKILHTNDIHGRLAEEKGRVIGMAKLKTVKEQEKPDLILDAGDAFQGLPLSNQSKGEEMAKAMNAVGYDAMAVGNHEFDFGYDQLKKLEGMLDFPMLSTNVYKDGKRAFKPSTIVTKNGIRYGIIGVTTPETKTKTRPEGIKGVEFRDPLQSVTAEMMRIYKDVDTFVVISHLGIDPSTQETWRGDYLVKQLSQNPQLKKRITVIDGHSHTVLQNGQIYNNDALAQTGTALANIGKVTFNYRNGEVSNIKPSLINVKDVENVTPNKALAEQINQADQTFRAQTAEVIIPNNTIDFKGERDDVRTRETNLGNAITDAMEAYGVKNFSKKTDFAVTNGGGIRASIAKGKVTRYDLISVLPFGNTIAQIDVKGSDVWTAFEHSLGAPTTQKDGKTVLTANGGLLHISDSIRVYYDMNKPSGKRINAIQILNKETGKFENIDLKRVYHVTMNDFTASGGDGYSMFGGPREEGISLDQVLASYLKTANLAKYDTTEPQRMLLGKPAVSEQPAKGQQGSKGSESGKDTQPIGKDKVMNPAKQPATGKVVLLPTHRGTVSSGAEGSDCALEGTAVSSKSGKQLTKMSASKGSGHEKQLPKTGTNQSSSPAAIFVLVAGIGLIATVRRRKAS

(19) United States
(12) Patent Application Publication
(10) Pub. No.: US 2023/0390373 A1
(43) Pub. Date: Dec. 7, 2023

(54) A LIVE STRAIN OF STAPHYLOCOCCUS AUREUS AND USES THEREOF

(71) Applicant: Versitech Limited, Hong Kong (CN)

(72) Inventors: Jiandong HUANG, Hong Kong (CN); Kwok Yung YUEN, Hong Kong (CN); Baozhong ZHANG, Hong Kong (CN); Jian DENG, Hong Kong (CN); Hin CHU, Hong Kong (CN)

(21) Appl. No.: 17/927,139

(22) PCT Filed: May 21, 2021
(86) PCT No.: PCT/CN2021/095165

§ 371 Date: Nov. 22, 2022

Related U.S. Application Data
(60) Provisional application No. 63/028,710, filed on May 22, 2020.
(60) Provisional application No. 63/123,635, filed on Dec. 10, 2020.

(51) Int. Cl. A61K 39/085; C12N 1/36; C12N 1/20

(52) U.S. Cl. CPC A61K 39/085; C12Y 205/01063; C12N 1/36; C12N 1/205; C12R 2001/445

(57) ABSTRACT

The invention relates to the field of biomedicine. In particular, the invention relates to a live strain of Staphylococcus aureus and uses thereof. More particularly, the invention relates to a live strain of Staphylococcus aureus which lacks adenosine synthase A (AdsA) activity, to a vaccine against Staphylococcus aureus infection comprising said live strain, and a method for preventing and/or treating Staphylococcus aureus infection in a subject by administering said live strain.

### REFERENCE TO SEQUENCE LISTING SUBMITTED ELECTRONICALLY

A sequence listing electronically submitted as an ASCII text file named P2021TC1576_ST25.txt, created on May 9, 2023 and having a size of 16000 bytes, is incorporated herein by reference in its entirety.

### TECHNICAL FIELD

The invention relates to the field of biomedicine. In particular, the invention relates to a live strain of Staphylococcus aureus and uses thereof. More particularly, the invention relates to a live strain of Staphylococcus aureus which lacks adenosine synthase A (AdsA) activity, to a vaccine against Staphylococcus aureus infection comprising said live strain, and a method for preventing and/or treating Staphylococcus aureus infection in a subject by administering said live strain.

### BACKGROUND

S. aureus is one of the most common causes of community-acquired (CA) and healthcare-associated (HA) bacterial infections (1). S. aureus infection leads to a variety of clinical manifestations ranging from skin and soft-tissue infections to invasive disease including bloodstream infection, endocarditis or sepsis (2). Moreover, the emergence of Methicillin-Resistant S. aureus (MRSA) has further made it a major global health problem (3). Of note is that prior exposure to S. aureus does not confer protection against subsequent S. aureus infection (4). The lack of understanding about how S. aureus constrains protective immunity has impeded the development of efficient treatments against S. aureus infection.

Several host factors to date have been implicated in the protection against S. aureus in different infection models. These include complement system, neutrophils (5), macrophages (6), IL-17A producing γδ+ T cells (7), humoral responses (8), Th1 and Th17 immune responses (6, 9). Nevertheless, humoral responses have long been recognized as a critical indicator of anti-S. aureus immunity, individuals with robust S. aureus specific antibody responses are not exempt from the next infection. In contrast, accumulating evidence has shed light on the role of cellular immunity in preventing the course of S. aureus infection. Patients with disease causing defect in Th17 differentiation often displayed increased susceptibility toward S. aureus infection (10). Th17 immunity can potentiate bacterial killing by enhancing phagocytosis of neutrophils via secreting IL-17 family cytokines (IL-17A and IL-17F) (11). Meanwhile memory Th1 immunity is reported to accelerate the clearance of S. aureus in blood stream infection (BSI) (6). Thus, the large number of recurrent infections in clinical setting implies failure in the establishment of protective T cell responses during S. aureus infection. Thus far, only O-acetyltransferase (OatA) has been proven to suppress the development of protective Th17 immunity by interfering with Th development cytokines milieu (12). Consequently, it is of significant importance to investigate the mechanisms whereby S. aureus counteracts host cellular immunity, contributing to reinfection.

Adenosine synthase A (AdsA) is an important virulence factor by which S. aureus modulates host pro-inflammatory responses, resulting in persistent infection (22). Previous studies have shown that AdsA can inhibit phagocytic clearance (23), secretion of antibacterial peptide sPLA2-IIA (24), and induce apoptosis of macrophages (25) via adenosine signaling or deoxyadenosine signaling. However, the mechanistic interaction of AdsA with host adaptive immunity remains unclear.

### SUMMARY OF THE INVENTION

In one aspect, the invention provides a vaccine against Staphylococcus aureus infection comprising a live strain of S. aureus, and optionally an adjuvant, wherein the strain lacks adenosine synthase A (AdsA) activity.

In another aspect, the invention provides a live strain of S. aureus for use in preventing and/or treating Staphylococcus aureus infection, wherein the strain lacks adenosine synthase A (AdsA) activity.

In another aspect, the invention provides a method for preventing and/or treating Staphylococcus aureus infection in a subject, which comprises administering an effective amount of a live strain of S. aureus to the subject, wherein the strain lacks adenosine synthase A (AdsA) activity.

In another aspect, the invention provides use of a live strain of S. aureus in preparation of a medicament for preventing and/or treating Staphylococcus aureus infection, wherein the strain lacks adenosine synthase A (AdsA) activity.

In another aspect, the invention provides a kit for immunization against S. aureus infection, comprising a container containing the vaccine of the invention or the live strain of S. aureus of the invention.

### DESCRIPTION OF THE Drawings

FIG. 1. S. aureus adsA mutant strain elicits potent inflammatory responses. (A) Survival of BALB/c mice infected with wild-type USA300 strain or adsA mutant strain [10₇ CFU, iv (intravenously)] for 14 days (n=10 mice per group; **P<0.005, Kaplan-Meier survival analysis). Data are representative of two replicative experiments. (B) ELISA analysis of TNF-α, IL-6, IL-1β in blood collected from BALB/c mice infected with wild-type USA300 strain or adsA mutant strain (10⁷ CFU, iv, 3 hours post-infection) (n=5 mice per group; *P<0.05, ***P<0.001, Mann Whitney U test). (C) qPCR analysis of TNF-α, IL-6, IL-1β and IFN-γ mRNA expression in tissues (liver, lung, spleen) harvested from BALB/c mice infected with wild-type USA300 strain or ΔadsA (10⁷ CFU, iv, 24 hours post-infection) (n=3; *P<0.05, **P<0.005, ***P<0.001, ns P>0.05, Student's t test). (D) qPCR analysis of TNF-α, IL-6, IL-1β and IFN-γ mRNA expression in HMDM infected by wild-type USA300 strain or adsA mutant strain (MOI=100; 8 hours) (n=3; *P<0.05, **P<0.005, ***P<0.001, Student's t test). Data are representative of three independent experiments. All data are shown as means±SD.

FIG. 2. Staphylococcus aureus inhibits inflammasome activation via adsA and adenosine production. (A) Analysis of cell viability changes in HMDM after infection with wild-type USA300 strain or ΔadsA strain (MOI=100; 3 to 8 hours) (n=3; *P<0.05,**P<0.005, Student's t test). (B) Analysis of cell viability changes in HMDM after infection with wild-type USA300 strain or adsA mutant strain (MOI=100; 8 hours) in the absence or presence of indicated drugs [Z-VADFMK, 25 μM; VX765, 20 μM; Necrosulfonamide (NSA), 5 μM] (n=3; *P<0.05,**P<0.005, ns P>0.05, Student's t test). (C) Analysis of LDH release and IL-1β release in PBMC and HMDM after infection (MOI=100; 6 hours) (n=3; *P<0.05, **P<0.005, Student's t test). (D) Western blot analysis of indicated proteins in the supernatant (SN) or cell lysate in PBMC and HMDM after infection with wild-type USA300 strain or adsA mutant strain (MOI=100; 6 hours). (E) Analysis of IL-1β release and immunoblot of indicated proteins in the supernatant (SN) or cell lysate in BMDC after infection with wild-type USA300 strain or adsA mutant strain (MOI=20; 6 hours) (n=3; ***P<0.0001, Student's t test). (F) Analysis of IL-1β release and immunoblot of indicated proteins in the supernatant (SN) or cell lysate in BMDC after infection with adsA variant strain (MOI=20; 6 hours) in the presence or absence of adenosine (100 μM) (n=3; ***P<0.001, Student's t test). Data are representative of two independent experiments. All data are shown as means±SD.

FIG. 3. Adenosine synthase A dampens NLRP3 inflammasome mediated IL-1β release via A2a receptor. (A) Analysis of IL-1β release in BMDC infected with wild-type USA300 strain or adsA mutant strain (MOI=20; 6 hours) in the absence or presence of indicated drugs (MCC950, 10 μM; VX765, 10 μM) (n=3; ns P>0.05, ***P<0.001, Student's t test). (B) Analysis of IL-1β release in BMDC infected with wild-type USA300 strain or ΔadsA strain (MOI=20; 6 hours) in the absence or presence of indicated drug (Cytochalasin D, 5 μM) (n=3; ***P<0.001, Student's t test). (C) Analysis of IL-1β release after siRNA-mediated knockdown of Aim2, Nlrp3 and Asc in BMDC infected with wild-type USA300 strain or ΔadsA strain (MOI=20; 6 hours) (n=3; ns P>0.05, *P<0.05, **P<0.005, Student's t test). (D) Analysis of IL-1β release in BMDC infected with wild-type USA300 strain or ΔadsA strain (MOI=20; 6 hours) in the absence or presence of indicated drug (ZM241385, 10 μM) (n=3; ***P<0.001, Student's t test). (E) qPCR analysis of NLRP3 expression in indicated BMDC infected adsA mutant strain (MOI=5 or 10; 4 hours) in the absence or presence of adenosine (100 μM) (n=3; **P<0.005, Student's t test). (F) qPCR analysis of NLRP3 expression in indicated BMDC treated with LPS (100 ng/ml) or Pam₃CSK₄ (100 ng/ml) for 4 hours in the absence or presence of adenosine (100 μM) (n=3; **P<0.005, Student's t test). Data are representative of two independent experiments. All data are shown as means±SD. (G) Immunoblot analysis of NLRP3 protein level in BMDC pre-treated with a series of dose of adenosine and stimulated with LPS (100 ng/ml) for 4 hours.

FIG. 4. Adenosine synthase A inhibits DC maturation and perturbs cytokines milieu for optimal T cell immunity. (A) Flow cytometric analysis of expression of maturation markers including MHC-II, CD86 and CD40 in BMDC infected with wild-type USA300 strain or ΔadsA strain (MOI=10; 16 hours) (n=3; *P<0.05, **P<0.005, Student's t test). (B) qPCR analysis of IL-1β, IL-6, IL-23, TGF-β, IL-12 and IL-10 mRNA expression in BMDC infected with wild-type USA300 strain or adsA mutant strain (MOI=10; 8 hours) (n=3; *P<0.05, ***P<0.001, ns P>0.05, Student's t test). (C) ELISA analysis of IL-1β, IL-6, IL-23 and IL-12 in culture supernatant collected from BMDC infected with wild-type USA300 strain or adsA mutant strain (MOI=10; 8 hours) (n=3; *P<0.05, **P<0.005, ***P<0.001, Student's t test). Data are representative of two independent experiments. All data are shown as means±SD.

FIG. 5. Adenosine synthase A restrains Th17 response via NLRP3 inflammasome and A2aR pathway. (A) Schematic graph for S. aureus intraperitoneal reinfection model. (B) Survival of 3× re-infected BALB/c mice re-challenged with wild-type USA300 strain [4×10⁷ CFU, iv (intravenously)] for 14 days (n=10 mice per group; *P<0.05, Kaplan-Meier survival analysis). (C) ELISA analysis of IL-17A and IFN-γ in cultures supernatant from splenocytes (harvested from 3× infection mice) re-stimulated by heat-killed S. aureus USA300 (MOI=5, 4 days) (n=6 mice per group; **P<0.005, Mann-Whitney U test). (D) ELISA analysis of IL-17A and IFN-γ in cultures supernatant from splenocytes (harvested from 1× infection mice) re-stimulated by heatkilled S. aureus USA300 (MOI=5, 4 days) (n=6 mice per group; **P<0.005, Mann-Whitney U test). (E) ELISA analysis of mean total and S. aureus specific IgG levels in the serum from mice re-infected with wild-type USA300 strain or adsA variant (4×10⁷ CFU, i.p.) at day 17 (n=8 mice per group; **P<0.005, ***P<0.001, Mann-Whitney U test). (F) Indirect ELISA analysis of S. aureus specific IgG levels in the serum collected at day 7, 14, 28 and 56 from mice 1× infected with wild-type USA300 strain or adsA mutant strain (4×10⁷ CFU, i.p.) (n=6 mice per group). (G) CFU analysis of staphylococcal burden in kidneys from 3× re-infected BALB/c mice which were re-challenged with wild-type USA300 strain for three days (2×10⁷ CFU, iv) (n=6 mice per group; **P<0.005, Mann-Whitney U test). (H) Representative kidney H&E-stained histologic sections at 3 days in re-infection model. (I) ELISA analysis of IL-17A in cultures supernatant from splenocytes (harvested from 3× infection mice treated with indicated inhibitors and vehicle control) re-stimulated by heat-killed S. aureus USA300 (MOI=5, 4 days) (n=5 mice per group; *P<0.05, **P<0.005, ***P<0.001, ANOVA followed by Bonferroni correction). Data are representative of two independent experiments. All data are shown as means±SD.

FIG. 6. Staphylococcal burden in blood and tissue. (A) CFU analysis of staphylococcal burden in blood from BALB/c mice infected with wild-type USA300 strain or its isogenic adsA mutant (10₇ CFU, iv, 3 hours post-infection) (n=10 mice per group; *P<0.05, Mann-Whitney U test). (B) CFU analysis of staphylococcal burden in blood from BALB/c mice infected with wild-type USA300 strain or its isogenic adsA mutant (10⁷ CFU, iv, 6 hours post-infection) (n=10 mice per group; **P<0.005, Mann-Whitney U test). (C) CFU analysis of staphylococcal burden in lung from BALB/c mice infected with wild-type USA300 strain or its isogenic adsA mutant (10⁷ CFU, iv, 24 hours post-infection) (n=6 mice per group; ns P>0.05, Mann-Whitney U test). (D) CFU analysis of staphylococcal burden in spleen from BALB/c mice infected with wild-type USA300 strain or its isogenic adsA mutant (10⁷ CFU, iv, 24 hours post-infection) (n=6 mice per group; ns P>0.05, Mann-Whitney U test). All data are shown as means±SD.

FIG. 7. S. aureus inhibit inflammasome activation in THP1. (A) Analysis of LDH release, IL-1β release and immunoblot of indicated proteins in the supernatant (SN) or cell lysate in THP1 derived macrophages after infection with wild-type USA300 strain or ΔadsA strain (MOI=50; 6 hours) (n=3; ***P<0.001, Student's t test). All data are shown as means±SD.

FIG. 8. Analysis of siRNA-mediated knock down in BMDC. (A) qPCR analysis of Aim2, Nlrp3, Asc expression in indicated BMDC treated with indicated siRNA for 48 hours. (B) Analysis of IL-1β release after sgRNA-mediated knockout of AIM2, NLRP3 and ASC in THP-1 derived macrophages infected with wild-type USA300 strain or ΔadsA strain (MOI=50; 6 hours) (n=3; **P<0.005, ***P<0.001, Student's t test).

FIG. 9. Survival condition of mice in reinfection model. (A) Survival analysis of BALB/c mice intraperitoneally re-infected with wild-type USA300 strain, adsA mutant strain or mixture of these two strains (4×10⁷ CFU, iv) for 14 days (n=5 mice per group; *P<0.05, Kaplan-Meier survival analysis) Data are representative of two replicative experiments.

FIG. 10. Schematic summary of AdsA in the modulation of NLRP3 mediated IL-1β release and Th17 differentiation.

### DETAILED DESCRIPTION

Before the aspects of the present invention are described, it must be noted that as used herein and in the appended claims, the singular forms “a”, “an”, and “the” include plural reference unless the context clearly dictates otherwise. The term “and/or” is intended to encompass any combinations of the items connected by this term, equivalent to listing all the combinations individually. For example, “A, B and/or C” encompasses “A”, “B”, “C”, “A and B”, “A and C”, “B and C”, and “A and B and C”. Unless defined otherwise, all technical and scientific terms used herein have the same meanings as commonly understood by one of ordinary skill in the art to which this invention belongs.

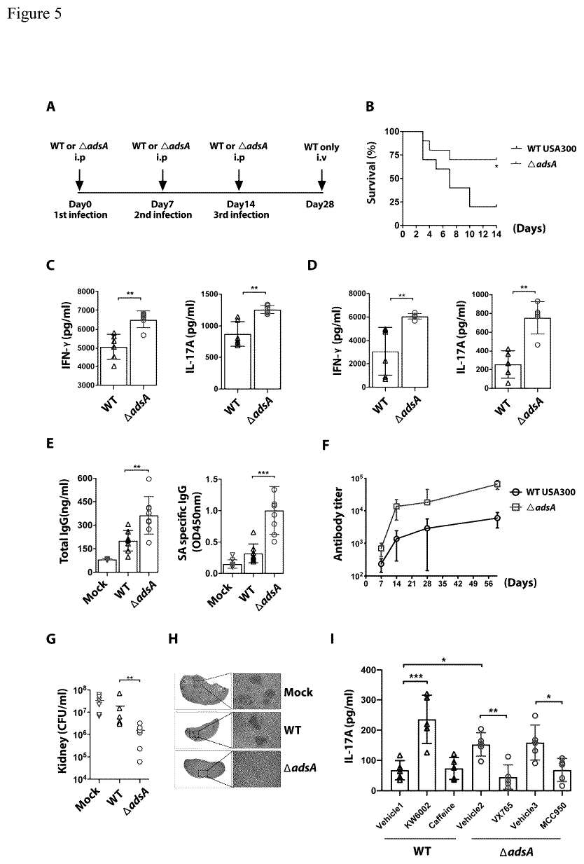

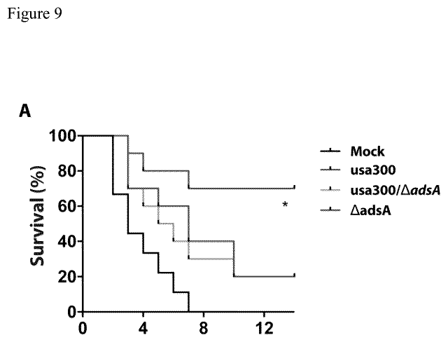

Staphylococcus aureus is a common human pathogen, capable of causing diverse illnesses with possibility of recurrent infections, and adenosine synthase A (AdsA) is a potent S. aureus virulence factor. The present inventors surprisingly found that a live strain of S. aureus lacking AdsA activity can protect mice against wildtype S. aureus infection (see such as, Example 5, FIG. 5 and FIG. 9).

Accordingly, in one aspect, the invention provides a vaccine against Staphylococcus aureus infection comprising a live strain of S. aureus, wherein the strain lacks adenosine synthase A (AdsA) activity.

Adenosine synthase A (AdsA) is an important virulence factor of S. aureus. An exemplary AdsA of S. aureus comprises an amino acid sequence of SEQ ID NO:46. But it is well known to a person skilled in the art that the AdsA of S. aureus may have minor differences from SEQ ID NO:46 due to polymorphyism between strains, while retain the same or similar functions.

SEQ ID NO 46:
MKALLLKTSVWLVLLFSAMGLWQVSSAAEQHTPMKAHAVTTIDKATTDRQLVLPTKEAAHOSGEEA
ATNVSASAQGTADDTNNKVTSNAPSNKPSTAVSTTVNETHDVDAQQASTQKPTQSATFKLSNAKTASLS
PRMFAANAPQTTTHKILHTNDIHGRLAEEKGRVIGMAKLKTVKEQEKPDLILDAGDAFQGLPLSNOSKG
EEMAKAMNAVGYDAMAVGNHEFDFGYDQLKKLEGMLDFPMLSTNVYKDGKRAFKPSTIVTKNGIRYGII
GVTTPETKTKTRPEGIKGVEFRDPLQSVTAEMMRIYKDVDTFVVISHLGIDPSTQETWRGDYLVKQLSQ
NPQLKKRITVIDGHSHTVLONGQIYNNDALAQTGTALANIGKVTFNYRNGEVSNIKPSLINVKDVENVT
PNKALAEQINQADQTFRAQTAEVIIPNNTIDFKGERDDVRTRETNLGNAITDAMEAYGVKNFSKKTDFA
VTNGGGIRASIAKGKVTRYDLISVLPFGNTIAQIDVKGSDVWTAFEHSLGAPTTQKDGKTVLTANGGLL
HISDSIRVYYDMNKPSGKRINAIQILNKETGKFENIDLKRVYHVTMNDFTASGGDGYSMFGGPREEGIS
LDQVLASYLKTANLAKYDTTEPQRMLLGKPAVSEQPAKGOOGSKGSESGKDTQPIGKDKVMNPAKQPAT
GKVVLLPTHRGTVSSGAEGSDCALEGTAVSSKSGKOLTKMSASKGSGHEKQLPKTGTNQSSSPAAIFVL
VAGIGLIATVRRRKAS

In some embodiments, the live strain of S. aureus comprises a deletion of an AdsA gene encoding AdsA. The AdsA gene may be completely deleted from the S. aureus strain so that no AdsA protein is present in the strain. The AdsA gene may also be partially deleted so that merely a truncated AdsA protein without activity is present in the strain, for example, at least a portion of AdsA responsible for adenosine production is deleted.

In some embodiments, the live strain of S. aureus comprises a mutation in an AdsA gene encoding AdsA. Such a mutation can be addition, substitution, or deletion of one or more nucleotides. In some embodiments, said mutation is a frame-shift mutation, which results in mistranslation of the AdsA protein.

In some embodiments, the mutation in the AdsA gene results in a deletion of a portion of AdsA responsible for adenosine production.

In some embodiments, the AdsA activity is responsible for attenuation of NLRP-3 mediated IL-1β production in an inflammatory cell via the adenosine/A2AR pathway during Staphylococcus aureus infection.

Preferably, the deletion of the AdsA gene is carried out by means of a strategy that avoids the reversal of the mutated strain to the wild phenotype.

In some embodiments, the strategy chosen to prevent the reversal of the mutated strain to the wild phenotype is the double homologous recombination.

In some embodiments, the mutation/deletion of the AdsA gene is carried out by targeted mutation, such as via CRISPR, TALEN or ZFN technologies.

In some embodiments, the vaccine may further comprise an adjuvant. As used herein, “adjuvant” refers to additional components in a vaccine to enhance the immune response, or ancillary molecules added to the vaccine or generated by the body after the respective induction by such additional components, like but not restricted to interferons, interleukins or growth factors. “Adjuvants” as used herein, can include aluminum hydroxide and aluminum phosphate, saponins, water-in-oil emulsion, oil-in-water emulsion, water-in-oil-in-water emulsion.

In some embodiments, the vaccine further comprises a pharmaceutically acceptable carrier. As used herein, “pharmaceutically acceptable carrier” includes any and all solvents, dispersion media, coatings, antifungal agents, isotonic and absorption delaying agents, and the like that are physiologically compatible. Non-limiting examples of pharmaceutically acceptable carriers include water, NaCl, physiological saline, lactated Ringer's, normal sucrose, normal glucose, binders, fillers, disintegrants, lubricants, coatings, sweeteners, flavoring agents, salt solutions (such as Ringer's solution), alcohol, oil, gelatin, carbohydrates such as lactose, amylose or starch, fatty acid esters, hydroxymethyl cellulose, polyvinylpyrrolidone and coloring agents.

The live strain of S. aureus can be derived from any S. aureus strains, such as those well known in the art. For example, the live strain of the invention may be derived from Staphylococcus aureus USA300, Newman, ATCC29213, and the like.

In some embodiments, the Staphylococcus aureus infection is a skin infection, soft-tissue infection, or invasive disease. In some embodiments, the invasive disease is bloodstream infection, endocarditis or sepsis.

In some embodiments, the Staphylococcus aureus infection is methicillin-resistant S. aureus (MRSA) infection or methicillin-sensitive S. aureus (MSSA) infection. In some preferred embodiments, the infection is a recurring S. aureus infection.

In some embodiments, the vaccine is formulated in a form for intramuscular administration, intraperitoneal administration, subcutaneous administration, oral administration or intranasal administration. In one embodiment, the vaccine is not for intravenous administration.

In some embodiments, the vaccine is in a lyophilized form, which can be reconstituted before use.

In another aspect, the invention provides a live strain of S. aureus for use in preventing and/or treating Staphylococcus aureus infection, wherein the strain lacks adenosine synthase A (AdsA) activity.

In some embodiments, the strain of S. aureus comprises a deletion of an AdsA gene encoding AdsA.

In some embodiments, the strain of S. aureus comprises a mutation in an AdsA gene encoding AdsA.

In some embodiments, the mutation in the AdsA gene results in a deletion of a portion of AdsA responsible for adenosine production.

In some embodiments, the AdsA activity is responsible for attenuation of NLRP-3 mediated IL-1β production in an inflammatory cell via the adenosine/A2AR pathway during Staphylococcus aureus infection.

In some embodiments, the strain is derived from Staphylococcus aureus USA300, Newman, or ATCC29213.

In some embodiments, the Staphylococcus aureus infection is a skin infection, soft-tissue infection, or invasive disease.

In some embodiments, the invasive disease is bloodstream infection, endocarditis or sepsis.

In some embodiments, the Staphylococcus aureus infection is methicillin-resistant S. aureus (MRSA) infection or methicillin-sensitive S. aureus (MSSA) infection. In some preferred embodiments, the infection is a recurring S. aureus infection.

In some embodiments, the strain is administered intramuscularly, intraperitoneally, subcutaneously, orally or intranasally. In one embodiment, the strain is not for intravenous administration.

In some embodiments, the live strain is in a lyophilized form, which can be reconstituted before use.

In another aspect, the invention provides a method for preventing and/or treating Staphylococcus aureus infection in a subject, which comprises administering an effective amount of a live strain of S. aureus to the subject, wherein the strain lacks adenosine synthase A (AdsA) activity.

As used herein, “effective amount” refers to an amount of a substance, compound, material, or composition containing a compound (such as the live strain of the invention of the vaccine of the invention) which is at least sufficient to produce a therapeutic effect after administration to a subject. Therefore, it is an amount necessary to prevent, cure, improve, retard or partially retard the symptoms of a disease or disorder, such as S. aureus infection.

The actual dosage of the live strain or vaccine of the present invention to be administered to a subject can be determined according to the following physical and physiological factors: weight, sex, severity of symptoms, type of diseases to be treated, previous or current therapeutic intervention, unknown etiological disease of the patient, administration time, administration route and the like. In any case, the amount of the live strains in the vaccine and the appropriate dose for an individual subject will be determined by the medical personnel responsible for administration.

In some embodiments, the strain of S. aureus comprises a deletion of an AdsA gene encoding AdsA.

In some embodiments, the strain of S. aureus comprises a mutation in an AdsA gene encoding AdsA.

In some embodiments, the mutation in the AdsA gene results in a deletion of a portion of AdsA responsible for adenosine production.

In some embodiments, the AdsA activity is responsible for attenuation of NLRP-3 mediated IL-1β production in an inflammatory cell via the adenosine/A2AR pathway during Staphylococcus aureus infection.

In some embodiments, the strain is derived from Staphylococcus aureus USA300, Newman, or ATCC29213.

In some embodiments, the Staphylococcus aureus infection is a skin infection, soft-tissue infection, or invasive disease.

In some embodiments, the invasive disease is bloodstream infection, endocarditis or sepsis.

In some embodiments, the Staphylococcus aureus infection is methicillin-resistant S. aureus (MRSA) infection or methicillin-sensitive S. aureus (MSSA) infection. In some preferred embodiments, the infection is a recurring S. aureus infection.

In some embodiments, the strain is administered intramuscularly, intraperitoneally, subcutaneously, orally or intranasally. In one embodiment, the strain is not administered intravenously.

In some embodiments, the strain is in a lyophilized form, which can be reconstituted before use.

In another aspect, the invention provides use of a live strain of S. aureus in preparation of a medicament for preventing and/or treating Staphylococcus aureus infection, wherein the strain lacks adenosine synthase A (AdsA) activity.

In some embodiments, the live strain of S. aureus comprises a deletion of an AdsA gene encoding AdsA.

In some embodiments, the live strain of S. aureus comprises a mutation in an AdsA gene encoding AdsA.

In some embodiments, the mutation in the AdsA gene results in a deletion of a portion of AdsA responsible for adenosine production.

In some embodiments, the AdsA activity is responsible for attenuation of NLRP-3 mediated IL-1β production in an inflammatory cell via the adenosine/A2AR pathway during Staphylococcus aureus infection.

In some embodiments, the strain is derived from Staphylococcus aureus USA300, Newman, or ATCC29213.

In some embodiments, the Staphylococcus aureus infection is a skin infection, soft-tissue infection, or invasive disease.

In some embodiments, the invasive disease is bloodstream infection, endocarditis or sepsis.

In some embodiments, the Staphylococcus aureus infection is methicillin-resistant S. aureus (MRSA) infection or methicillin-sensitive S. aureus (MSSA) infection. In some preferred embodiments, the infection is a recurring S. aureus infection.

In some embodiments, the live strain of S. aureus is in the form for intramuscular administration, intraperitoneal administration, subcutaneous administration, oral administration, or intranasal administration.

In some embodiments, the live strain is in a lyophilized form, which can be reconstituted before use.

In another aspect, the invention provides a kit for immunization against S. aureus infection, comprising a container containing the vaccine of the invention or the live strain of S. aureus of the invention.

In another aspect, the invention provides a method of enhancing IL-1β production and/or Th1/Th17 responses by inhibiting A2a receptor.

In another aspect, the invention provides a method to downregulate S. aureus-specific Th1/Th17 responses by inhibiting NLRP3 and/or caspase-1.

### EXAMPLES

A further understanding of the present invention may be obtained by reference to the specific examples set forth herein, which are only intended to illustrate the invention, and are not intended to limit the scope of the invention. It is apparent that various modifications and variations may be made to the present invention without departing from the spirit of the invention, and such modifications and variations are therefore also within the scope of the present invention.

### Materials and Methods

### Reagents and Primers

The Reagents and Primers as used are described in Table 1 below.

TABLE 1
Reagents and Primers

| REAGENT | SOURCE | IDENTIFIER |
|---|---|---|
| Antibodies | | |
| Rabbit monoclonal antibody to pro- and | Abcam | ab179515 |
| cleaved caspase-1 | | |
| Mouse monoclonal antibody to B-actin | Sigma Aldrich | A5316 |
| Anti-Mouse I-A/I-E FITC | BD Biosciences | cat. 553623 |
| Anti-Mouse CD86 PE | BD Biosciences | cat. 553692 |
| Anti-Mouse CD40 PE | BD Biosciences | cat. 553791 |
| Chemicals and others | | |
| Adenosine | Sigma Aldrich | A4036 |
| Cytochalasin D | Sigma Aldrich | C2618 |
| ZM241385 | Sigma Aldrich | Z0153 |
| Caffeine | Sigma Aldrich | C1778 |
| Z-VAD-FMK | MedChemExpress | HY-16658B |
| VX765 | MedChemExpress | HY-13205 |
| Necrosulfonamide | MedChemExpress | HY-100573 |
| MCC950 | InvivoGen | inh-mcc |
| Ultrapure LPS, E. coli 0111: B4 | InvivoGen | tlrl-3pelps |
| Pam₃CSK₄ | InvivoGen | tlrl-pms |
| Phorbol 12-myristate 13-acetate (PMA) | InvivoGen | tlrl-pma |
| KW6002 | Tocris | 5147 |
| PEI-MAX | Polysciences | 24765-1 |
| Ficoll-Paque Plus | GE Healthcare | 17144002 |
| Red cell lysis buffer | BioLegend | 420301 |
| Lipofectamine ® RNAiMAX | Invitrogen | 13778030 |
| TRIzol Reagent | Invitrogen | 15596026 |
| hGM-CSF | PeproTech | 300-03 |
| mGM-CSF | PeproTech | 315-03 |
| Commercial assay kits | | |
| CellTiter-Glo ® Luminescent Cell Viability | Promega | G7570 |
| Assay kit | | |
| CytoTox 96 ® Non-Radioactive Cytotoxicity | Promega | G1780 |
| Assay kit | | |
| ELISA MAX ™ Standard Set Mouse IL-1β | BioLegend | 432601 |
| ELISA MAX ™ Standard Set Mouse IL-6 | BioLegend | 431301 |
| ELISA MAX ™ Deluxe Set Mouse IL-23 | BioLegend | 433704 |
| ELISA MAX ™ Deluxe Set Mouse IL-12 | BioLegend | 433604 |
| ELISA MAX ™ Deluxe Set Mouse IL-17A | BioLegend | 432504 |
| ELISA MAX ™ Deluxe Set Mouse IFN-γ | BioLegend | 430804 |
| ELISA MAX ™ Deluxe Set Mouse TNF-α | BioLegend | 430904 |
| Human IL-1β ELISA kit | R&D Systems | DLB50 |
| Mouse IgG ELISA Kit | Abcam | ab157719 |
| SYBR Premix Ex Taq kit | Ta Ka Ra | RR820A |
| PVDF membranes | Sigma Aldrich | GE10600069 |
| Primers | SEQ ID NO | |
| siRNA for Aim2: GAUAGAGUACUGUAUGGUATT | SEQ ID NO: 1 | |
| siRNA for Nlrp3 1: UCUCAAGUCUAAGCACCAATT | SEQ ID NO: 2 | |
| siRNA for Nlrp3 2: CAUCAAUGCUGCUUCGACATT | SEQ ID NO: 3 | |
| siRNA for Nlrp3 3: CAGUGACAAUACUCUGGGATT | SEQ ID NO: 4 | |
| siRNA for Asc 1: GAGCAGCUGCAAACGACUATT | SEQ ID NO: 5 | |
| siRNA for Asc 2: GCUACUAUCUGGAGUCGUATT | SEQ ID NO: 6 | |
| siRNA for Asc 3: CCUGGAACCUGACCUGCAATT | SEQ ID NO: 7 | |
| Il-1β F_primer: GCAACTGTTCCTGAACTCAACT | SEQ ID NO: 8 | |
| Il-1β R_primer: ATCTTTTGGGGTCCGTCAACT | SEQ ID NO: 9 | |
| Il-6 F_primer: TAGTCCTTCCTACCCCAATTTCC | SEQ ID NO: 10 | |
| Il-6 R_primer: TTGGTCCTTAGCCACTCCTTC | SEQ ID NO: 11 | |
| Il-23 F_primer: ATGCTGGATTGCAGAGCAGTA | SEQ ID NO: 12 | |
| Il-23 R_primer: ACGGGGCACATTATTTTTAGTCT | SEQ ID NO: 13 | |
| Il-12 F_primer: TGGTTTGCCATCGTTTTGCTG | SEQ ID NO: 14 | |
| Il-12 R_primer: ACAGGTGAGGTTCACTGTTTCT | SEQ ID NO: 15 | |
| Il-10 F_primer: GCTCTTACTGACTGGCATGAG | SEQ ID NO: 16 | |
| Il-10 R_primer: CGCAGCTCTAGGAGCATGTG | SEQ ID NO: 17 | |
| Tgf-β F_primer: CTCCCGTGGCTTCTAGTGC | SEQ ID NO: 18 | |
| Tgf-β R_primer: GCCTTAGTTTGGACAGGATCTG | SEQ ID NO: 19 | |
| Tnf-α F_primer: CAGGCGGTGCCTATGTCTC | SEQ ID NO: 20 | |
| Tnf-α R_primer: CGATCACCCCGAAGTTCAGTAG | SEQ ID NO: 21 | |
| Ifn-γ F_primer: GCCACGGCACAGTCATTGA | SEQ ID NO: 22 | |
| Ifn-γ R_primer: TGCTGATGGCCTGATTGTCTT | SEQ ID NO: 23 | |
| Nlrp3 F_primer: TGGATGGGTTTGCTGGGAT | SEQ ID NO: 24 | |
| Nlrp3 R_primer: CTGCGTGTAGCGACTGTTGAG | SEQ ID NO: 25 | |
| Gapdh F_primer: TCACCACCATGGAGAAGGC | SEQ ID NO: 26 | |
| Gapdh R_primer: GCTAAGCAGTTGGTGGTGCA | SEQ ID NO: 27 | |
| Human IL-1β F primer: | SEQ ID NO: 28 | |
| ATGATGGCTTATTACAGTGGCAA | | |
| Human IL-1β R primer: | SEQ ID NO: 29 | |
| GTCGGAGATTCGTAGCTGGA | | |
| Human IL-6 F primer: | SEQ ID NO: 30 | |
| ACTCACCTCTTCAGAACGAATTG | | |
| Human IL-6 R primer: | SEQ ID NO: 31 | |
| CCATCTTTGGAAGGTTCAGGTTG | | |
| Human TNF-α F primer: | SEQ ID NO: 32 | |
| CCTCTCTCTAATCAGCCCTCTG | | |
| Human TNF-α R primer: | SEQ ID NO: 33 | |
| GAGGACCTGGGAGTAGATGAG | | |
| Human IFN-γ F_primer: | SEQ ID NO: 34 | |
| TCGGTAACTGACTTGAATGTCCA | | |
| Human IFN-γ R primer: | SEQ ID NO: 35 | |
| TCGCTTCCCTGTTTTAGCTGC | | |
| Human GAPDH F primer: | SEQ ID NO: 36 | |
| ACAACTTTGGTATCGTGGAAGG | | |
| Human GAPDH R primer: | SEQ ID NO: 37 | |
| GCCATCACGCCACAGTTTC | | |
| sgRNA for AIM2 F primer: | SEQ ID NO: 38 | |
| CACCGGCAAGATATTATCGGCACAG | | |
| sgRNA for AIM2 R primer: | SEQ ID NO: 39 | |
| AAACCTGTGCCGATAATATCTTGCC | | |
| sgRNA for NLRP3 F primer: | SEQ ID NO: 40 | |
| CACCGCTGATTAGTGCTGAGTACCG | | |
| sgRNA for NLRP3 R primer: | SEQ ID NO: 41 | |
| AAACCGGTACTCAGCACTAATCAGC | | |
| sgRNA for ASC F primer: | SEQ ID NO: 42 | |
| CACCGCGAGGGTCACAAACGTTGAG | | |
| sgRNA for ASC R primer: | SEQ ID NO: 43 | |
| AAACCTCAACGTTTGTGACCCTCGC | | |
| sgRNA for caspase-1 F primer: | SEQ ID NO: 44 | |
| CACCGCTTTAAACCACACCACACCA | | |
| sgRNA for caspase-1 R primer: | SEQ ID NO: 45 | |
| AAACTGGTGTGGTGTGGTTTAAAGC | | |

### Cell culture

THP1 were purchased from the American Type Culture Collection (ATCC) and cultured in RPMI-1640 supplemented with 10% heat-inactivated fetal bovine serum (HI-FBS), 100 U/ml penicillin and 0.1 mg/ml streptomycin. Before infection experiment, THP1 were differentiated into macrophages with 50 nM Phorbol 12-myristate 13-acetate (PMA) for 24 hours. After stimulation, cells were washed with 1640-RPMI medium and cultured with medium without PMA for 24 hours.

### Human Primary Cell Culture

Human peripheral blood mononuclear cells (PBMC) were isolated from human buffy coat (provided by Department of Microbiology, The University of Hong Kong, Li Ka Shing Faculty of Medicine) by Ficoll-Paque gradient protocol. In brief, 30 mL of 1:1 PBS diluted buffy coat from healthy donors were layered on Ficoll-Paque Plus (GE Healthcare, Life Sciences) and centrifuged at 450×g for 30 min at room temperature. Separated layers of PBMC were collected and then washed 2 times with RPMI-1640 medium. After washing, the cells were resuspended in 4 mL red blood cell lysing buffer (Biolegend, RBC Lysis Buffer) and incubated for 5 min at room temperature. Following two subsequent washes, the cell pellet was resuspended in RPMI-1640 media supplemented with 10% FBS, 100 U/ml penicillin and 0.1 mg/ml streptomycin for further infection experiments. To differentiate human monocytes-derived macrophages (HMDM), isolated PBMC were seeded on poly-L-lysine coated coverslips in 24 well plate and cultured in RPMI-1640 media supplemented with L-glutamine, 10% FBS, 1×penicillin/streptomycin, 10 mM HEPES, 50 ng/mL hGM-CSF (PeproTech) for up to 7 days differentiation.

### Mice Dendritic Cells Culture

Bone marrow cells extracted from femur of 8-12 weeks old female BALB/c mice were culture in RPMI-1640 medium supplemented with L-glutamine, 10% heat inactivated-FBS, 1×penicillin/streptomycin, 10 mM HEPES, 50 μM-β mercaptoethanol, 20 ng/ml mGM-CSF (PeproTech) for up to 7 days differentiation.

### Bacterial Strains

S. aureus strains USA300 and its isogenic adsA variant were grown in Brain Heart Infusion (BHI) at 37° C. Unmarked, non-polar deletion of adsA was constructed using plasmid pKOR1 as described previously (26). Briefly, 5′- and 3′-flanking regions of adsA was PCR amplified from chromosomal DNA of S. aureus strain USA300 with primers adsA-UF (5′ CGGAATTCTGCGGCTCATGCAATGAC 3′), adsA-UR (5′ GGCACTGACATGTTCGAGACTTGCCATAATC 3′), adsA-DF (5′ AGTCTCGAACATGTCAGTGCCTAAAGGTAG 3′), adsA-DR (5′ GGGGTACCTCCCTACAGCTAAAATGG 3′) and the individual PCR products were mixed to generate an in-frame deletion pattern of adsA. The overlapping amplicon containing the in-frame deletion pattern was sub-cloned into pKOR1, to generate pKOR1-ΔadsA. The recombinant plasmid pKOR1-ΔadsA was firstly introduced into DH5a, followed by electro-transformed into S. aureus RN4220 and subsequently into USA300. The selection of allelic replacement was performed as described previously, and the deletion of adsA was further confirmed by PCR using primers adsA-UF/adsA-DR and inner primers adsA-IF (5′ TATCCATGGCCGACTAGC 3′)/adsA-IR (5′ ACCTGTTTGTGCCAATGC 3′) specific for the deleted sequence and DNA sequencing.

### Animals

All animals care and experiments were performed in accordance with the Association for Assessment and Accreditation of Laboratory Animal Care guidelines (www.aaalac.org) and with approval from our institutional animal care and use committee. BALB/c mice were provided from the Laboratory Animal Unit of the University of Hong Kong. Mice were housed in specific-pathogen free facilities and 8 to 12-week old female mice were utilized for all in vitro and in vivo experiments.

### Cell Infection Experiments

One day before bacterial infection experiments, S. aureus strains were inoculated and cultured with BHI broth for overnight. Next day, overnight culture of bacteria strains were sub-cultured in fresh BHI broth at a dilution of 1:100 and grown at 37° C. Following 3 hours of culturing, S. aureus were harvested and washed for two to three times in cold PBS by centrifugation. Finally, S. aureus strains were diluted with desired volume of PBS, yielding an OD600 of 0.5 (1×10⁸ CFU/ml), and further centrifuged and resuspended at desired bacterial concentration. The number of bacteria was determined by serial dilution and colony formation on BHI agar plates. Mammalian cells were plated in 24-well plates at a number of 4×10⁵ per well and infected with S. aureus strains in antibiotic free medium at the indicated MOI.

### Animal Infection Experiments

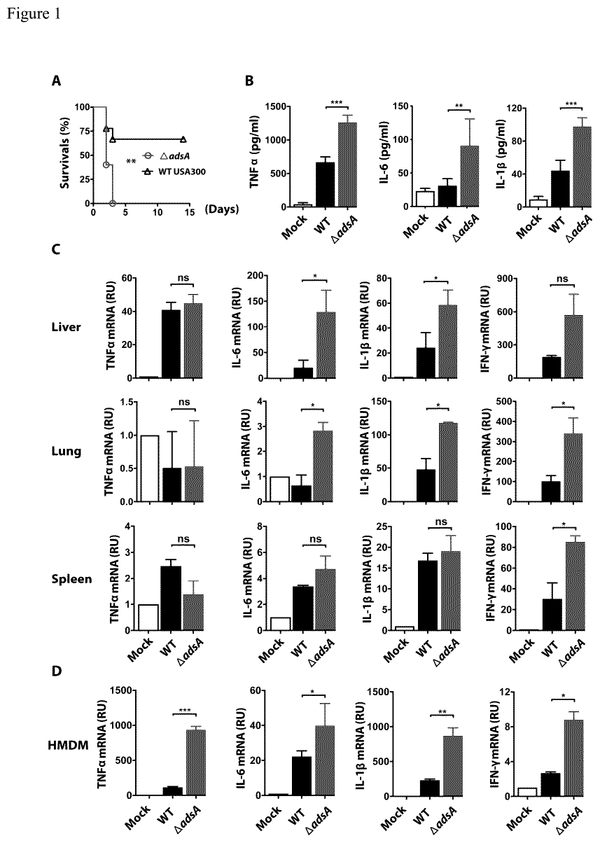

Protocol for harvest and calculation of wild type and variant S. aureus strains was the same as described above. To induce systemic blood infection model of S. aureus in FIG. 1, 200 ul of bacterial suspension (1×10⁷ CFU) was administered intravenously into naïve 6-wk-old female BALB/c mice. To establish re-infection model in FIG. 5, mice were intraperitoneally infected with WT S. aureus (USA300), adsA mutant strain or WT S. aureus (USA300)/adsA mutant strain (4×10⁷ CFU) for a total of 3 times at 7 days interval. After 14 days convalescent period, mice were re-challenged with lethal dose of WT USA300 S. aureus (4×10⁷ CFU) or sublethal dose of WT USA300 S. aureus (2×10⁷ CFU). Upon bacterial infection, health conditions of mice were frequently monitored in compliance with humane end points (HEP) form. To measure staphylococcal burden in blood, after 2 h of i.v. infection, mice were anaesthetized by intraperitoneal injection of 80-120 mg ketamine and 3-6 mg xylazine per kilogram of body weight and blood was collected via tail vein. Blood samples were incubated on ice in 0.5% saponin/PBS for lysis of host cells. Later on, serial dilutions were performed on BHI agar plates for colony formation. To enumerate bacteria in tissues, mice were euthanized by CO₂ inhalation, organs including lungs, spleens and kidneys were harvested and homogenized in 1% Triton X-100/PBS. Aliquots of homogenates were serially diluted and spread on BHI agar plates for colony formation. For histopathology, kidneys were incubated in 4% paraformaldehyde (PFA) at room temperature for 24 h. Tissues were embedded in paraffin, thin sectioned, stained with hematoxylin-eosin, and examined by microscopy.

### In Vitro Re-Stimulation of Splenocytes

Animals were sacrificed at indicated time points in re-infection model. Spleens were harvested and grinded for cells suspension. After centrifugation, splenocytes were experienced red blood cell lysing, washes and filtering, and single cells suspension was cultured in RPMI-1640 media supplemented with 10% FBS, 100 U/ml penicillin and mg/ml streptomycin. For re-stimulation, splenocytes were seeded in 24 well plates at 4×10⁵ cells/well and stimulated with heat-killed S. aureus at a MOI of 5 for 4 days. Culture supernatants were collected for measurement of cytokines by ELISA.

### BMDC Stimulation and Flow Cytometry Analysis

After differentiation, BMDC were plated in 24-well plates at a number of 4×10⁵ cells in each well and infected with S. aureus strains at the indicated MOI. For surface marker analysis, cells were detached with PBS containing 5 mM EDTA and were incubated in FACS buffer (PBS containing 3% FBS and 0.1% sodium azide). After incubation with purified neutralizing monoclonal antibodies against CD16:CD32 (Fc Block; Biolegend) for 15 minutes at 4° C., cells were staining with specific antibodies for 30 minutes at 4° C. in the dark. The following antibody were used for flow cytometry analysis: Anti-Mouse I-A/I-E FITC (cat. 553623; BD Biosciences), Anti-Mouse CD86 PE (cat. 553692; BD Biosciences), Anti-Mouse CD40 PE (cat. 553791; BD Biosciences). The stained cells were then analyzed using a flow cytometer (ACEA NovoCyte Quanteon) and FlowJo 10.4.0 software (TreeStar, Co).

### Cell Viability Assay and ELISA

Culture supernatants of relevant cells were collected and centrifuged at 13000 rpm for 4 min to get rid of cell debris and bacteria. Levels of LDH in culture supernatants were measured by CytoTox 96 Non-Radioactive Cytotoxicity Assay kit (Promega). Cell viability was measured by the CellTiter Glo Luminescent Cell Viability Assay (Promega). ELISA assay was conducted according manufactures' instructions.

### Generation of CRISPR-Cas9 Knockout Cell Lines

All THP1 knock-out cell lines in this study were generated by Cas9-encoding lentiCRISPRv2 vector from Zhang Feng lab (Addgene plasmid #52961). Single guide RNAs (sgRNAs) targeting human AIM2, NLRP3, PYCARD and caspase-1 were designed utilizing online sgRNA Designer from Broad Institute. All sgRNAs were annealed and cloned into plasmid lentiCRISPRv2 according to Zhang Feng's protocol.

The Lentiviral particles were produced from HEK293T cells transfected with lentiCRISPRv2 vector, and two packaging plasmids pMD2.G and psPAX2 (Addgene plasmids #12259 and #12260) using PEI-MAX (Polysciences) and were further concentrated by ultracentrifugation. THP-1 cells were transduced by spinoculation in the presence of 8 μg/mL polybrene. A polyclonal population was selected using 1 mg/ml puromycin for at least one week. Genetic ablation was verified by Western blot analysis.

### RNA Interference

All siRNAs were designed according to previous published studies and synthesized by by GenePharma (Shanghai, China). The control siRNA (negative control) was provided by GenePharma. Sequence of siRNAs were listed in Table 1. Lipofectamine® RNAiMAX Reagent (Invitrogen) were used for transient transfection of siRNAs into BMDC. 48-72 hours after transfection, BMDC were prepared for bacterial infection experiment.

### Western Blot

For detection of cleaved form of caspase-1, cell culture supernatants were precipitated by methanol-chloroform method. Briefly, supernatant was mixed with an equal volume of methanol and 0.25 volumes of chloroform, vortexed and centrifuge for 15 min at 20000 g. The upper phase was discarded and the interphase was mixed with methanol. After centrifugation for 5 min at 20000 g, the pellet was resuspended in 2×SDS-PAGE sample buffer and boiled for 5 min at 100° C. Protein samples were separated by 15% SDS-PAGE gels and were transferred onto PVDF membranes. Total cell lysates lysed by RIPA buffer (50 mM Tris-Cl, pH 7.4, 150 mM NaCl, 1% Triton X-100, 0.1% SDS, 1% sodium deoxycholate and 0.5 mM EDTA) supplemented with 1× protease inhibitor cocktail (Roche) were boiled for 5 min at 100° C. Lysate aliquots were separated by SDS-PAGE gels and transferred onto PVDF membranes. Blots were probed with primary antibody: rabbit anti-caspase-1 (1:1000 dilution, 179515 from Abcam, USA), mouse anti-β actin (1:5000 dilution, A5316 from Sigma). Anti-rabbit or mouse antibodies conjugated to HRP were used as secondary reagents.

### RNA Isolation and Quantitative Reverse Transcription PCR

Total RNA was extracted from cells using TRIzol Reagent (Invitrogen) and 1 μg total RNA was used for reverse transcription (Takara) according to manufactures' instructions. The cDNA was then used for quantitative RT-PCR to analyze relevant mRNA expression using Applied Biosystems StepOnePlus™ Real-Time PCR System and SYBR Premix Ex Taq kit (Takara) according to the manufacturer's instruction. Primers for interest of genes are listed in Table 1. The data were normalized to GAPDH and fold change in gene expression was calculated by comparative CT method (2^{-ΔΔCT}).

### Statistical Analysis

Data are presented as means±SD. Data from in vitro experiments were assumed to follow a normal distribution. Therefore, to compare means from two groups Unpaired Student's test was used. One-way analysis of variance (ANOVA) with Bonferroni correction was utilized to compare means among multiple groups. Data from in vitro experiments normally do not follow normal distribution. Accordingly, non-parametric Mann-Whitney U-test was used. For survival analysis, Log-rank test was used. All statistical tests were performed by GraphPad Prism 8.0 and Microsoft Excel. P value less than 0.05 was considered to be statistically significant.

### Example 1. S. aureus adsA Mutant Strain Elicits Potent Inflammatory Responses

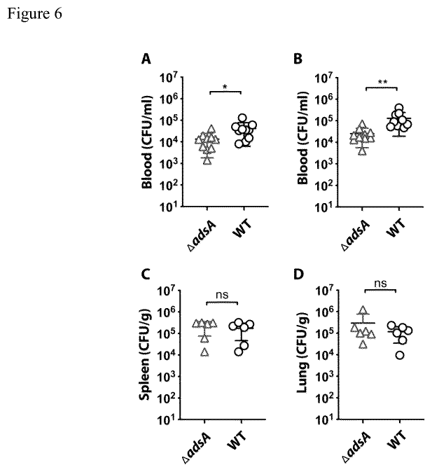

To explore the role of AdsA in modulating inflammatory responses, the inventors first generated an adsA mutant strain based on USA300 background by allelic replacement (26). BALB/c mice were then infected by intravenous (i.v.) injection with 107 CFU of wild-type S. aureus USA300 or its isogenic adsA variant. The survival of the mice was monitored for 14 days. Interestingly, 70% of mice infected with wild-type USA300 survived, whereas mice infected with adsA mutant Staphylococci had all died by day 3 post infection (FIG. 1A). To measure staphylococcal burden upon i.v. infection, blood samples were collected from infected mice at different time points. Consistent with previous work (23), mice infected with adsA mutant displayed enhanced bacterial clearance in the blood (FIG. 6A, B). The inventors then speculated that AdsA may constrain excessive inflammatory responses upon invasive S. aureus infection and mice infected with adsA mutant may die from cytokines storm. To examine the proposed role of adsA in inhibiting innate immune responses during S. aureus infection, the level of common inflammatory cytokines was measured either in blood or tissues. Enzyme-linked immunosorbent assay (ELISA) showed that the production of TNF-α, IL-6 and IL-1β in blood was significantly higher in mice infected with adsA mutant strain when compared to those infected with wild type strain (FIG. 1B). In addition, quantitative real-time polymerase chain reaction (qRT-PCR) analysis indicated that the mRNA expression of IL-6, IL-1β was up-regulated in liver and lung, and IFN-γ was up-regulated in lung and spleen in response to adsA variant infection (FIG. 1C). And these effects were not caused by the difference of bacterial load in tissues, since CFUs recovered from two groups had no difference (FIG. 6C, D). In agreement with the observations in mice, the mRNA levels of TNF-α, IL-6, IL-1β and IFN-γ were up-regulated in human monocytes derived macrophages (HMDM) infected with adsA mutant as compared with the wild type strain (FIG. 1D). Collectively, these findings suggest that AdsA suppresses the production of pro-inflammatory cytokines during systemic S. aureus infection.

### Example 2. S. aureus Inhibits Inflammasome Activation via AdsA and Adenosine Production

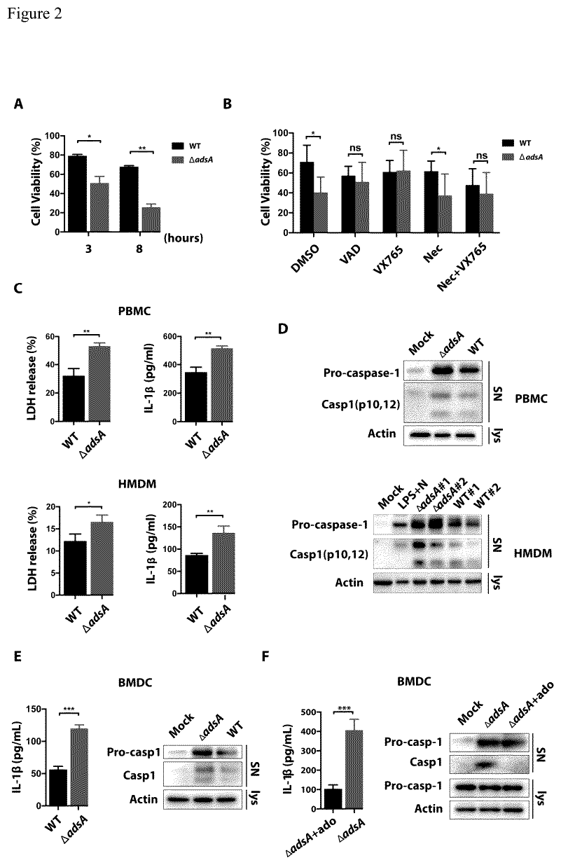

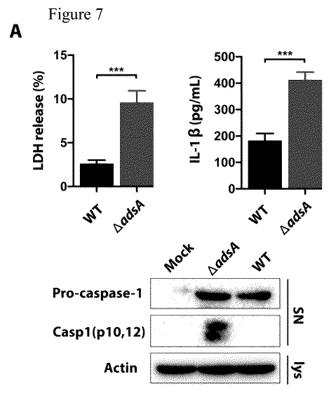

There are two major biological roles of inflammasome: (i) the maturation and secretion of a potent inflammatory cytokine, IL-1β and (ii) induction of pyroptosis (16). In mice intravenous infection model, adsA mutant strain evidently improved the production of IL-1β in blood, implying that AdsA might suppress the activity of inflammasome. To examine the effect of AdsA on inflammasome, the inventors measured the viability of HMDM after infection with either S. aureus USA300 or its isogenic adsA variant. The cell viability assay showed that adsA mutant significantly triggered cell death after 8 hours post infection, whereas 70% of HMDM infected by wild type strain remained alive (FIG. 2A). To further dissect the type of cell death (apoptosis, pyroptosis or necroptosis) induced by the mutant strain, pharmacological inhibitors were used to block corresponding molecular executors of cell death signaling. It is demonstrated that treatment of pan-caspase inhibitor, Z-VAD-FMK or caspase-1 inhibitor, VX765 can dramatically decrease adsA mutant-induced cell death, suggesting a specific role of AdsA in attenuating caspase-1 activity during S. aureus infection (FIG. 2B). To further determine the effect of AdsA on caspase-1 dependent inflammasome activation, Lactate dehydrogenase (LDH) release assay, ELISA of IL-1β and immunoblot analysis of caspase-1 activation were performed. The results showed that AdsA prevented pyroptosis and production of IL-1β in HMDM and peripheral blood mononuclear cells (PBMC) (FIG. 2C). Immunoblot analysis displayed that wild type strain impeded caspase-1 activation (yielding p10 and p12 band) in human macrophages and monocytes as compared with adsA variant (FIG. 2D). Similar results were also obtained in THP-1 derived macrophages (FIG. 7A). Activation of inflammasome in different type of cells can initiate different immune effects. Dendritic cells (DC) serve as a central bridge to adaptive immunity, the inventors next explored the role of AdsA in regulating IL-1β release in dendritic cells. Likewise, the results displayed that AdsA suppressed IL-1β release and caspase-1 cleaveage in mouse bone marrow derived dendritic cells (BMDC) (FIG. 2E). It is reported that AdsA can facilitate adenosine production by catalyzing degradation of adenosine monophosphate (AMP) (23). Adenosine is a physiologically immune modulator which inhibits secretion of proinflammatory cytokines, yet its role in IL-1β production during S. aureus infection is unclear. The inventors then speculated that wild type S. aureus may suppress inflammasome activation by adenosine. As expected, IL-1β release and immunoblot analysis showed that BMDC infected with adsA mutant displayed impaired inflammasome activation when pretreated with adenosine (FIG. 2F). Taken together, these results demonstrate that AdsA attenuates caspase-1 dependent inflammasome activation and IL-1β secretion in various type of immune cells, suggesting its broad effects on host immunity in the pathogenesis of S. aureus infection.

### Example 3. AdsA Dampens NLRP3 Inflammasome Mediated IL-1β Release Via Adenosine/A2AR Axis

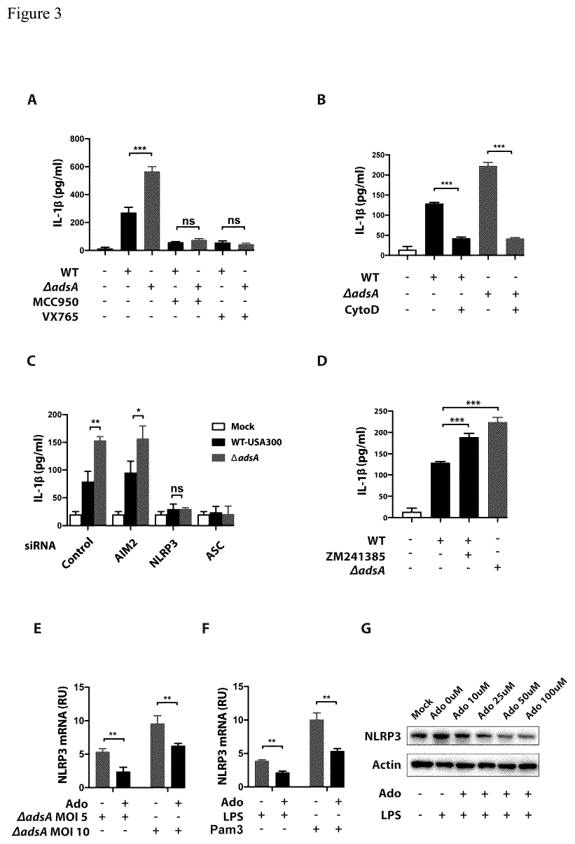

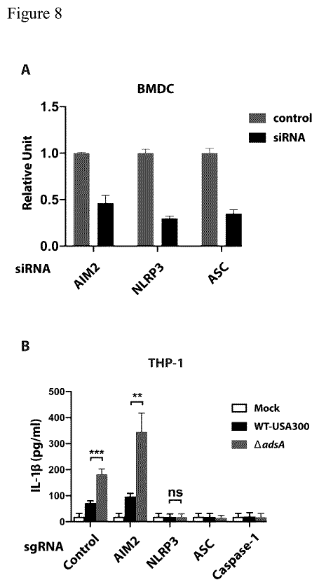

Since DC are professional antigen-presenting cell and critical mediator in initiating T lymphocytes lineage differentiation, inflammasome activation in DC could have profound influence on cellular immunity. The inventors therefore sought to delineate the detailed mechanism by which AdsA attenuates inflammasome activation in BMDC with pharmacological inhibitors and siRNA-mediated knockdown studies. Previous report demonstrated that phagocytosis linked PGN degradation is essential to NLRP3 inflammasome activation during S. aureus infection (20). To determine whether NLRP3 inflammasome is affected by AdsA, BMDC infected with wild-type or adsA mutant S. were treated with NLRP3 specific inhibitor MCC950. The results showed that IL-1β release in BMDC during S. aureus infection is primarily induced by NLRP3 inflammasome, as inhibition of NLRP3 can largely dampen IL-1β production to the level similar to caspase-1 inhibition by VX765 (FIG. 3A). And consistent with previous studies, pretreatment with Cytochalasin D, a potent inhibitor of actin polymerization and phagocytosis, nearly suppressed IL-1β production in BMDC (FIG. 3B). Considering that MCC950 almost prevented the release of IL-1β from BMDC infected by S. aureus regardless of adsA mutation, only NLRP3 inflammasome responds to live S. aureus stimulation in vitro infection assays, implying a role of AdsA in affecting NLRP3 inflammasome. To rule out potential off-target effect of pharmacological inhibitors, the inventors next conducted infection experiments in BMDC with knockdown of AIM2 (absent in melanoma 2, intracellular DNA sensing inflammasome), NLRP3 and ASC (PYD and CARD domain containing, which is a common downstream molecule of AIM2 and NLRP3 inflammasome) by siRNA treatment (FIG. 8A). The results showed that IL-1β production was significantly decreased in BMDC with knockdown of NLRP3 and ASC instead of AIM2, further confirming that only NLRP3 inflammasome is activated upon S. aureus infection (FIG. 3C). The similar result was also observed in THP-1 derived macrophages which are ablated of AIM2, NLRP3, ASC and caspase-1 individually by CRISPR-Cas9 editing (FIG. 8B).

The immune modulatory characteristics of adenosine are attributed to four trans-membrane receptors: A1, A2A, A2B, and A3 (27). Activation of these receptors can induce pro-inflammatory or anti-inflammatory effect, and the abundance and distribution of four receptors varies in different cell types and tissues. Among them, A2A receptor (A2AR) is known for its anti-inflammatory trait in immune cells. Hence, the inventors try to figure out whether AdsA/adenosine/A2AR signaling is implicated in NLRP3 inflammasome in dendritic cells. The result showed that addition of ZM241385, a pharmacological inhibitor of A2AR improved IL-1β production in BMDC infected with wild type S. aureus to a level comparable to adsA mutant infection (FIG. 3D). To identify whether declined production of IL-1β by adenosine/A2AR signaling was because of influence on priming signal of NLRP3 inflammasome, we examined adenosine treatment decreased the expression level of NLRP3 in BMDC either activated by adsA mutant S. aureus or TLR2 and TLR4 agonist (Pam3CSK4 and LPS) (FIG. 3E, F). Moreover, adenosine also inhibited NLRP3 expression at protein level in a dose-dependent manner (FIG. 3G). Taken together, the results suggested that AdsA can specifically inhibit NLRP3 inflammasome activation via adenosine/A2AR axis in dendritic cells.

### Example 4. AdsA Inhibits DC Maturation and Perturbs Cytokines Milieu for Optimal T Cell Immunity

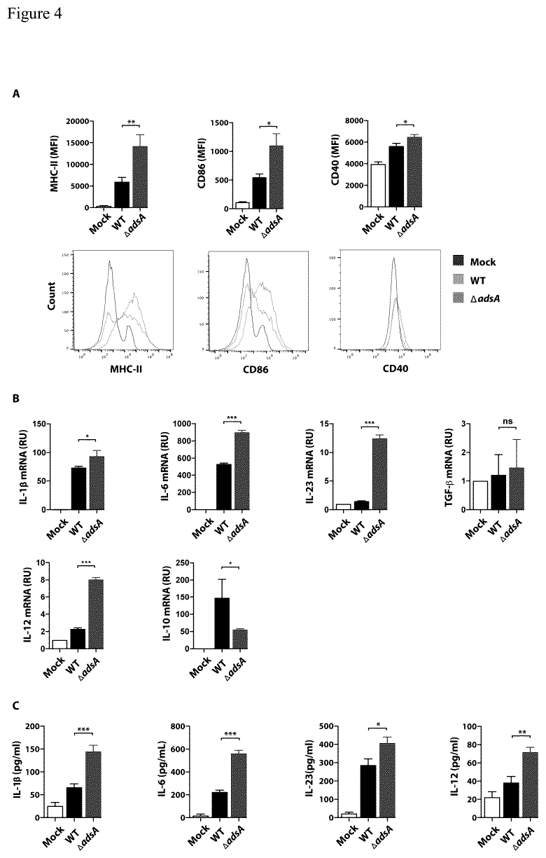

Increasing evidence showed that inflammasome helps to establish adaptive immunity through promoting production of danger signals or bioactive cytokines. Among them, IL-1β can manipulate extensive immune responses through IL-1R signaling by paracrine or autocrine. Since AdsA can inhibit IL-1β production in BMDC, the inventors next sought to characterize the role of AdsA in modulating function of dendritic cells, especially the cytokines environment for developing protective immunity. The inventors first evaluated the activation and maturation of BMDC under in vitro infection condition. Flow cytometry analysis showed that BMDC infected with adsA variant displayed higher expression of DC maturation markers including CD40, CD86, and major histocompatibility complex (MHC) II in comparison with wild type strain (FIG. 4A). Furthermore, mRNA expression and ELISA analysis showed that AdsA inhibited production of Th17-polarizing cytokines including IL-1β, IL-6 and IL-23 with the exception of TGF-β in S. aureus infected BMDC (FIGS. 4B & C). Besides, IL-10, an anti-inflammatory cytokine reported to inhibit Th17 development (29) was evidently up-regulated in BMDC incubated with wild type strain (FIG. 4B). Intriguingly, the inventors also found that Th1-polarizing cytokine IL-12 was significantly induced in BMDC infected with adsA mutant at the mRNA and protein levels (FIGS. 4B & C). Overall, the data indicate that Adenosine synthase A can hinder maturation of dendritic cells and suppress production of Th polarizing cytokines, implying a role of AdsA in coordinating subsequent Th1/Th17 immunity.

### Example 5. AdsA Suppresses Th17 Responses Via NLRP3 Inflammasome and A2AR Pathway In Vivo

To assess the influence of AdsA on adaptive immunity, the inventors adopted a murine reinfection model described elsewhere (6). In this model, BALB/c mice were repeatedly infected by intraperitoneal injection with wild-type S. aureus USA300 or its isogenic adsA variant. Eventually, mice in both groups were re-challenged with a lethal or sublethal dose of wild type S. aureus USA300 (FIG. 5A). Given that adsA mutant could be rapidly cleared by innate immunity upon systemic infection, the inventors designed a group of mice which were infected with a mixture of wild type and mutant strains. Intriguingly, the survival rate of mice repeatedly infected with adsA mutant is higher than that of mice re-infected with the wild type or the wild type/adsA mutant (FIG. 5B) (FIG. 9A). The results indicated that prior exposure to adsA mutant can confer better protection to host against subsequent S. aureus infection. In other words, wild type S. aureus could suppress the establishment of protective immunity via AdsA. To examine whether AdsA has an influence on Th1/Th17 responses, spleens from re-infected mice at day 7 and 20 (1× and 3× infection) were harvested and re-stimulated with heat-killed S. aureus for 4 days. ELISA analysis of splenocytes culture showed that mice re-infected with adsA mutant had increased production of IL-17A and IFN-γ compared to the wild type, indicating an enhanced Th17 and Th1 responses in these mice (FIGS. 5C & D). As for humoral responses, adsA variant re-infection induced higher levels of total IgG and S. aureus specific IgG in the serum in comparison with wild type re-infection group (FIG. 5E). The same results of antibody responses were also observed in lx infected mice (FIG. 5F). Additionally, the immune response elicited by adsA mutant confers improved protection to mice, as the bacterial load in kidneys of mice re-infected with adsA mutant was significantly lower than that in wild type group upon sublethal S. aureus challenge (FIG. 5G). By histology, kidneys from mice in mock and wild type group developed more and larger abscesses as compared with those adsA mutant re-infected mice (FIG. 5H).

The role of inflammasome/IL-1β/IL-1R signaling in the development of antigen specific Th17 responses is well defined (17, 28). And our in vitro experiments using BMDC implied that AdsA dampens NLRP3 inflammasome mediated IL-1β release via A2A receptor (FIG. 3E). The inventors therefore attempt to examine whether adenosine/A2AR/NLRP3 axis is participated in Th17 development during the course of S. aureus infection using in vivo model. In line with in vitro infection experiments, mice pretreated with A2AR specific antagonist developed stronger Th17 responses than vehicle control when infected with wild type S. aureus. However, treatment of caffeine, a pan adenosine receptor antagonist, did not have any influence on Th17 immunity (FIG. 50. Furthermore, mice pretreated with caspase-1 inhibitor, VX765 or NLRP3 inhibitor, MCC950 showed decreased IL-17A production in comparison with their vehicle control upon wild type S. aureus infection, indicating a role of NLRP3 inflammasome in the differentiation of S. aureus specific Th17 immunity. Taken together, the inventors demonstrate that AdsA suppresses Th17 immunity in vivo via adenosine/A2AR/NLRP3 axis, causing recurrent S. aureus infection.

### Discussion

Staphylococcus aureus is characteristic of its capability of evading host immunity, resulting in persistent infection and recurrent infection (4). In particular, subversion from T cell responses was reported to be critical in recurrent S. aureus infection (6, 12). In this study, the inventors demonstrate that AdsA can suppress the production of proinflammatory cytokines which is important for the development of protective T cell responses. Mechanistically, this study also highlights the role of AdsA in the evasion of host protective Th17 immunity by impairing NLRP3 inflammasome mediated IL-1β release via adenosine/A2AR pathway. Our findings potentiate the understanding of host-pathogen interaction during S. aureus infection.

Being a vital intracellular sensor involved in host-pathogen interaction, inflammasome actively participates in the process of S. aureus pathogenesis (13). Mice deficient in inflammasome had decreased neutrophils recruitment, resulting in impaired bacterial clearance at the site of infection (21). It is well-established that NLRP3 inflammasome is activated in several S. aureus infection murine models. The underlying mechanisms can be divided into two aspects: (1) pore forming toxins (hemolysin, leukocidin and Panton-Valentine leukocidin) produced by S. aureus cause rupture of cellular membrane, leading to potassium efflux which is recognized as a common mechanism for NLRP3 inflammasome activation; (2) phagocytosis and lysosomal degradation of S. aureus peptidoglycan also contributes to NLRP3 inflammasome mediated IL-1β release (20, 29). In the present study, immune cells were stimulated by live S. aureus instead of bacterial culture filtrates containing large amount of PFTs and BMDC treated with MCC950 or cytochalasin D had little IL-1β production, implying that phagocytosis dependent NLRP3 activation predominate in the present in vitro infection assays. The production of IL-1β was also reported to be regulated by RIP1/RIP3/MLKL mediated necroptosis, which constrains excessive inflammasome (30). However, in present work, treatment of necroptosis inhibitor before infection, necrosulfonamide (NSA), did not have apparent effect on S. aureus induced cytotoxicity. The potential explanations may lie in different infection conditions or cell types. The inventors' work also highlights a role of adenosine signaling in AdsA mediated IL-1β inhibition, as verified by adenosine and A2AR antagonist in vitro infection assays. The enzymatic activity of AdsA is well-defined, which can facilitate the degradation of ATP, ADP and AMP to adenosine (31) or conversion of neutrophil extracellular traps (NETs) to deoxyadenosine (25). The results do not exclude the possibility that AdsA may suppress inflammasome in vivo by other mechanisms. In murine model, bacterial infection can increase extracellular ATP levels and NLRP3 inflammasome activation, thereby promoting anti-bacterial immunity (32). Given that AdsA is capable of degrading extracellular ATP, it is possible that AdsA could suppress IL-1β production by decreasing ATP levels in vivo.

Mechanistically, the inventors also provide evidence that AdsA/adenosine/A2AR axis might affect S. aureus induced IL-1β release by interfering with priming signal of NLRP3 inflammasome. It is demonstrated that AdsA or adenosine can act on A2a receptor by inhibiting NF-κB and p38 MAPK activity, both of which were contributing to NLRP3 priming signal (12, 33). In contrast to present findings, other group reported that adenosine and A2a receptor signaling could enhance NLRP3 inflammasome activation by amplifying priming signal (34). In their study, BMDM were treated with adenosine after a long period of LPS priming, which is distinct from the infection conditions in the present study, indicating a complex role of adenosine in the regulation of inflammasome at different stages of bacterial infection. Therefore, the detailed mechanism of adenosine/A2AR axis in the modulation of NLRP3 inflammasome during S. aureus infection merits further investigation.

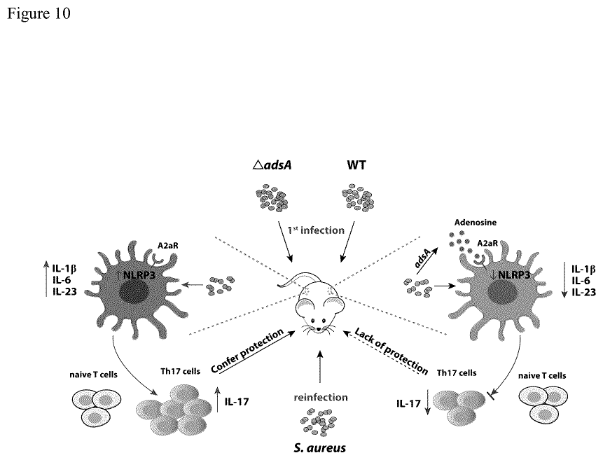

In summary, as shown in FIG. 10, the inventors' work highlights that S. aureus subverts protective immune responses by AdsA, leading to recurrent infection. The results also illuminate a mechanism that AdsA restricts Th17 immune responses via A2AR/NLRP3/IL-1β axis, improving survival of S. aureus in subsequent infection. Better understanding of how S. aureus evades from host immunity will facilitate the development of treatment and vaccination against S. aureus infection.

### REFERENCES

1. R. M. Klevens, J. R. Edwards, R. P. Gaynes, S. National Nosocomial Infections Surveillance, The impact of antimicrobial-resistant, health care-associated infections on mortality in the United States. Clinical infectious diseases: an official publication of the Infectious Diseases Society of America 47, 927-930 (2008).
2. M. Z. David, R. S. Daum, Community-associated methicillin-resistant Staphylococcus aureus: epidemiology and clinical consequences of an emerging epidemic. Clinical microbiology reviews 23, 616-687 (2010).
3. M. Yilmaz, N. Elaldi, Balkan, II, F. Arslan, A. A. Batirel, M. Z. Bakici, M. G. Gozel, S. Alkan, A. D. Celik, M. A. Yetkin, H. Bodur, M. Sinirtas, H. Akalin, F. A. Altay, I. Sencan, E. Azak, S. Gundes, B. Ceylan, R. Ozturk, H. Leblebicioglu, H. Vahaboglu, A. Mert, Mortality predictors of Staphylococcus aureus bacteremia: a prospective multicenter study. Annals of clinical microbiology and antimicrobials 7 (2016).
4. A. J. Kallen, Y. Mu, S. Bulens, A. Reingold, S. Petit, K. Gershman, S. M. Ray, L. H. Harrison, R. Lynfield, G. Dumyati, J. M. Townes, W. Schaffner, P. R. Patel, S. K. Fridkin, M. I. o. t. E. I. P. Active Bacterial Core surveillance, Health care-associated invasive MRSA infections, 2005-2008. Jama 304, 641-648 (2010).
5. A. N. Spaan, B. G. Surewaard, R. Nijland, J. A. van Strijp, Neutrophils versus Staphylococcus aureus: a biological tug of war. Annual review of microbiology 67, 629-650 (2013).
6. A. F. Brown, A. G. Murphy, S. J. Lalor, J. M. Leech, K. M. O'Keeffe, M. Mac Aogain, D. P. O'Halloran, K. A. Lacey, M. Tavakol, C. H. Hearnden, D. Fitzgerald-Hughes, H. Humphreys, J. P. Fennell, W. J. van Wamel, T. J. Foster, J. A. Geoghegan, E. C. Lavelle, T. R. Rogers, R. M. McLoughlin, Memory Th1 Cells Are Protective in Invasive Staphylococcus aureus Infection. PLoS Pathog 11, e1005226 (2015).
7. A. G. Murphy, K. M. O'Keeffe, S. J. Lalor, B. M. Maher, K. H. Mills, R. M. McLoughlin, Staphylococcus aureus infection of mice expands a population of memory gammadelta T cells that are protective against subsequent infection. J Immunol 192, 3697-3708 (2014).
8. C. P. Montgomery, M. Daniels, F. Zhao, M. L. Alegre, A. S. Chong, R. S. Daum, Protective immunity against recurrent Staphylococcus aureus skin infection requires antibody and interleukin-17A. Infection and immunity 82, 2125-2134 (2014).
9. L. Lin, A. S. Ibrahim, X. Xu, J. M. Farber, V. Avanesian, B. Baquir, Y. Fu, S. W. French, J. E. Edwards, Jr., B. Spellberg, Th1-Th17 cells mediate protective adaptive immunity against Staphylococcus aureus and Candida albicans infection in mice. PLoS pathogens 5, e1000703 (2009).
10. J. D. Milner, J. M. Brenchley, A. Laurence, A. F. Freeman, B. J. Hill, K. M. Elias, Y. Kanno, C. Spalding, H. Z. Elloumi, M. L. Paulson, J. Davis, A. Hsu, A. I. Asher, J. O'Shea, S. M. Holland, W. E. Paul, D. C. Douek, Impaired T (H)17 cell differentiation in subjects with autosomal dominant hyper-IgE syndrome. Nature 452, 773-776 (2008).
11. B. Stockinger, M. Veldhoen, Differentiation and function of Th17 T cells. Curr Opin Immunol 19, 281-286 (2007).
12. M. Sanchez, S. L. Kolar, S. Muller, C. N. Reyes, A. J. Wolf, C. Ogawa, R. Singhania, D. D. De Carvalho, M. Arditi, D. M. Underhill, G. A. Martins, G. Y. Liu, O-Acetylation of Peptidoglycan Limits Helper T Cell Priming and Permits Staphylococcus aureus Reinfection. Cell Host Microbe 22, 543-551 e544 (2017).
13. J. H. Melehani, J. A. Duncan, Inflammasome Activation Can Mediate Tissue-Specific Pathogenesis or Protection in Staphylococcus aureus Infection. Curr Top Microbiol Immunol 397, 257-282 (2016).
14. C. Dostert, V. Petrilli, R. Van Bruggen, C. Steele, B. T. Mossman, J. Tschopp, Innate immune activation through Nalp3 inflammasome sensing of asbestos and silica. Science (New York, N.Y.) 320, 674-677 (2008).
15. S. Mariathasan, D. S. Weiss, K. Newton, J. McBride, K. O'Rourke, M. Roose-Girma, W. P. Lee, Y. Weinrauch, D. M. Monack, V. M. Dixit, Cryopyrin activates the inflammasome in response to toxins and ATP. Nature 440, 228-232 (2006).
16. F. Martinon, K. Burns, J. Tschopp, The inflammasome: a molecular platform triggering activation of inflammatory caspases and processing of proIL-beta. Molecular cell 10, 417-426 (2002).
17. Y. Chung, S. H. Chang, G. J. Martinez, X. O. Yang, R. Nurieva, H. S. Kang, L. Ma, S. S. Watowich, A. M. Jetten, Q. Tian, C. Dong, Critical regulation of early Th17 cell differentiation by interleukin-1 signaling. Immunity 30, 576-587 (2009).
18. D. Holzinger, L. Gieldon, V. Mysore, N. Nippe, D. J. Taxman, J. A. Duncan, P. M. Broglie, K. Marketon, J. Austermann, T. Vogl, D. Foell, S. Niemann, G. Peters, J. Roth, B. Loffler, Staphylococcus aureus Panton-Valentine leukocidin induces an inflammatory response in human phagocytes via the NLRP3 inflammasome. Journal of leukocyte biology 92, 1069-1081 (2012).
19. J. H. Melehani, D. B. James, A. L. DuMont, V. J. Torres, J. A. Duncan, Staphylococcus aureus Leukocidin A/B (LukAB) Kills Human Monocytes via Host NLRP3 and ASC when Extracellular, but Not Intracellular. PLoS pathogens 11, e1004970 (2015).
20. T. Shimada, B. G. Park, A. J. Wolf, C. Brikos, H. S. Goodridge, C. A. Becker, C. N. Reyes, E. A. Miao, A. Aderem, F. Gotz, G. Y. Liu, D. M. Underhill, Staphylococcus aureus evades lysozyme-based peptidoglycan digestion that links phagocytosis, inflammasome activation, and IL-1beta secretion. Cell Host Microbe 7, 38-49 (2010).
21. J. S. Cho, Y. Guo, R. I. Ramos, F. Hebroni, S. B. Plaisier, C. Xuan, J. L. Granick, H. Matsushima, A. Takashima, Y. Iwakum, A. L. Cheung, G. Cheng, D. J. Lee, S. I. Simon, L. S. Miller, Neutrophil-derived IL-1beta is sufficient for abscess formation in immunity against Staphylococcus aureus in mice. PLoS Pathog 8, e1003047 (2012).
22. V. Thammavongsa, H. K. Kim, D. Missiakas, O. Schneewind, Staphylococcal manipulation of host immune responses. Nature reviews. Microbiology 13, 529-543 (2015).
23. V. Thammavongsa, J. W. Kern, D. M. Missiakas, O. Schneewind, Staphylococcus aureus synthesizes adenosine to escape host immune responses. The Journal of experimental medicine 206, 2417-2427 (2009).
24. E. Pernet, J. Brunet, L. Guillemot, M. Chignard, L. Touqui, Y. Wu, Staphylococcus aureus Adenosine Inhibits sPLA2-IIA-Mediated Host Killing in the Airways. J Immunol 194, 5312-5319 (2015).
25. V. Thammavongsa, D. M. Missiakas, O. Schneewind, Staphylococcus aureus degrades neutrophil extracellular traps to promote immune cell death. Science 342, 863-866 (2013).
26. T. Bae, O. Schneewind, Allelic replacement in Staphylococcus aureus with inducible counter-selection. Plasmid 55, 58-63 (2006).
27. K. A. Jacobson, Z. G. Gao, Adenosine receptors as therapeutic targets. Nature reviews. Drug discovery 5, 247-264 (2006).
28. A. Dunne, P. J. Ross, E. Pospisilova, J. Masin, A. Meaney, C. E. Sutton, Y. Iwakura, J. Tschopp, P. Sebo, K. H. Mills, Inflammasome activation by adenylate cyclase toxin directs Th17 responses and protection against Bordetella pertussis. J Immunol 185, 1711-1719 (2010).
29. A. J. Wolf, C. N. Reyes, W. Liang, C. Becker, K. Shimada, M. L. Wheeler, H. C. Cho, N. I. Popescu, K. M. Coggeshall, M. Arditi, D. M. Underhill, Hexokinase Is an Innate Immune Receptor for the Detection of Bacterial Peptidoglycan. Cell 166, 624-636 (2016).
30. K. Kitur, S. Wachtel, A. Brown, M. Wickersham, F. Paulino, H. F. Penaloza, G. Soong, S. Bueno, D. Parker, A. Prince, Necroptosis Promotes Staphylococcus aureus Clearance by Inhibiting Excessive Inflammatory Signaling Cell Rep 16, 2219-2230 (2016).
31. V. Thammavongsa, O. Schneewind, D. M. Missiakas, Enzymatic properties of Staphylococcus aureus adenosine synthase (AdsA). BMC Biochem 12, 56 (2011).
32. Y. Xiang, X. Wang, C. Yan, Q. Gao, S. A. Li, J. Liu, K. Zhou, X. Guo, W. Lee, Y. Zhang, Adenosine-5′-triphosphate (ATP) protects mice against bacterial infection by activation of the NLRP3 inflammasome. PLoS One 8, e63759 (2013).
33. F. G. Bauernfeind, G. Horvath, A. Stutz, E. S. Alnemri, K. MacDonald, D. Speert, T. Fernandes-Alnemri, J. Wu, B. G. Monks, K. A. Fitzgerald, V. Hornung, E. Latz, Cutting edge: NF-kappaB activating pattern recognition and cytokine receptors license NLRP3 inflammasome activation by regulating NLRP3 expression. J Immunol 183, 787-791 (2009).
34. X. Ouyang, A. Ghani, A. Malik, T. Wilder, O. R. Colegio, R. A. Flavell, B. N. Cronstein, W. Z. Mehal, Adenosine is required for sustained inflammasome activation via the A (2)A receptor and the HIF-1alpha pathway. Nat Commun 4, 2909 (2013).
35. C. L. Evavold, J. C. Kagan, How Inflammasomes Inform Adaptive Immunity. J Mol Biol 430, 217-237 (2018).
36. K. Shenderov, D. L. Barber, K. D. Mayer-Barber, S. S. Gurcha, D. Jankovic, C. G. Feng, S. Oland, S. Hieny, P. Caspar, S. Yamasaki, X. Lin, J. P. Ting, G. Trinchieri, G. S. Besra, V. Cemndolo, A. Sher, Cord factor and peptidoglycan recapitulate the Th17-promoting adjuvant activity of mycobacteria through mincle/CARD9 signaling and the inflammasome. J Immunol 190, 5722-5730 (2013).
37. M. Bruchard, G. Mignot, V. Derangere, F. Chalmin, A. Chevriaux, F. Vegran, W. Boireau, B. Simon, B. Ryffel, J. L. Connat, J. Kanellopoulos, F. Martin, C. Rebe, L. Apetoh, F. Ghiringhelli, Chemotherapy-triggered cathepsin B release in myeloid-derived suppressor cells activates the Nlrp3 inflammasome and promotes tumor growth. Nat Med 19, 57-64 (2013).
38. C. E. Zielinski, F. Mele, D. Aschenbrenner, D. Jarrossay, F. Ronchi, M. Gattorno, S. Monticelli, A. Lanzavecchia, F. Sallusto, Pathogen-induced human TH17 cells produce IFN-gamma or IL-10 and are regulated by IL-1beta. Nature 484, 514-518 (2012).
39. B. Z. Zhang, J. Cai, B. Yu, L. Xiong, Q. Lin, X. Y. Yang, C. Xu, S. Zheng, R. Y. Kao, K. Sze, K. Y. Yuen, J. D. Huang, Immunotherapy Targeting Adenosine Synthase A Decreases Severity of Staphylococcus aureus Infection in Mouse Model. J Infect Dis 216, 245-253 (2017).
40. B. Lee, R. Olaniyi, J. M. Kwiecinski, J. B. Wardenburg, Staphylococcus aureus toxin suppresses antigen-specific T cell responses. J Clin Invest 130, 1122-1127 (2020).

The foregoing description of the specific embodiments will so fully reveal the general nature of the disclosure that others can, by applying knowledge within the skill of the relevant art (s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art (s).

While various embodiments of the present disclosure have been described above, it should be understood that they have been presented by way of examples, and not limitation. It would be apparent to one skilled in the relevant art (s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A vaccine against Staphylococcus aureus infection comprising a live strain of S. aureus, and optionally an adjuvant or a pharmaceutically acceptable carrier, wherein the strain lacks adenosine synthase A (AdsA) activity.

2. The vaccine of claim 1, wherein the strain of S. aureus comprises a deletion of an AdsA gene encoding AdsA.

3. The vaccine of claim 1, wherein the strain of S. aureus comprises a mutation in an AdsA gene encoding AdsA.

4. The vaccine of claim 3, wherein the mutation in the AdsA gene results in a deletion of a portion of AdsA responsible for adenosine production.

5. The vaccine of claim 1, wherein the AdsA activity is responsible for attenuation of NLRP-3 mediated IL-1β production in an inflammatory cell via the adenosine/A2AR pathway during Staphylococcus aureus infection.

6. The vaccine of claim 1, wherein the strain is derived from Staphylococcus aureus USA300, Newman, or ATCC29213.

7. The vaccine of claim 1, wherein the Staphylococcus aureus infection is a skin infection, soft-tissue infection, or invasive disease.

8. The vaccine of claim 7, wherein the invasive disease is bloodstream infection, endocarditis or sepsis.

9. The vaccine of claim 1, wherein the Staphylococcus aureus infection is methicillin-resistant S. aureus (MRSA) infection or MSSA infection.

10. The vaccine of claim 1, wherein the vaccine is formulated in a form for intramuscular administration, intraperitoneal administration, subcutaneous administration, oral administration or intranasal administration, preferably, the vaccine is not for intravenous administration.

11. A live strain of S. aureus for use in preventing and/or treating Staphylococcus aureus infection, wherein the strain lacks adenosine synthase A (AdsA) activity.

12. A live strain of S. aureus for use according to claim 11, wherein the strain of S. aureus comprises a deletion of an AdsA gene encoding AdsA.

13. A live strain of S. aureus for use according to claim 12, wherein the strain of S. aureus comprises a mutation in an AdsA gene encoding AdsA.

14. A live strain of S. aureus for use according to claim 13, wherein the mutation in the AdsA gene results in a deletion of a portion of AdsA responsible for adenosine production.

15. A live strain of S. aureus for use according to claim 11, wherein the AdsA activity is responsible for attenuation of NLRP-3 mediated IL-1β production in an inflammatory cell via the adenosine/A2AR pathway during Staphylococcus aureus infection.

16. A live strain of S. aureus for use according to claim 11, wherein the strain is derived from Staphylococcus aureus USA300, Newman, or ATCC29213.

17. A live strain of S. aureus for use according to claim 11, wherein the Staphylococcus aureus infection is a skin infection, soft-tissue infection, or invasive disease.

18. A live strain of S. aureus for use according to claim 17, wherein the invasive disease is bloodstream infection, endocarditis or sepsis.

19. A live strain of S. aureus for use according to claim 11, wherein the Staphylococcus aureus infection is methicillin-resistant S. aureus (MRSA) infection or MSSA infection.

20. A live strain of S. aureus for use according to claim 11, wherein the strain is administered intramuscularly, intraperitoneally, subcutaneously, orally or intranasally, preferably, the live strain is not administered intravenously.

21. A method for preventing and/or treating Staphylococcus aureus infection in a subject, which comprises administering an effective amount of a live strain of S. aureus to the subject, wherein the strain lacks adenosine synthase A (AdsA) activity.

22. The method of claim 21, wherein the strain of S. aureus comprises a deletion of an AdsA gene encoding AdsA.

23. The method of claim 21, wherein the strain of S. aureus comprises a mutation in an AdsA gene encoding AdsA.

24. The method of claim 23, wherein the mutation in the AdsA gene results in a deletion of a portion of AdsA responsible for adenosine production.

25. The method of claim 21, wherein the AdsA activity is responsible for attenuation of NLRP-3 mediated IL-1β production in an inflammatory cell via the adenosine/A2AR pathway during Staphylococcus aureus infection.

26. The method of claim 21, wherein the strain is derived from Staphylococcus aureus USA300, Newman, or ATCC29213.

27. The method of claim 21, wherein the Staphylococcus aureus infection is a skin infection, soft-tissue infection, or invasive disease.

28. The method of claim 21, wherein the invasive disease is bloodstream infection, endocarditis or sepsis.

29. The method of claim 21, wherein the Staphylococcus aureus infection is methicillin-resistant S. aureus (MRSA) infection or MSSA infection.

30. The method of claim 21, wherein the strain is administered intramuscularly, intraperitoneally, subcutaneously, orally or intranasally, preferably, the strain is not administered intravenously.

31. Use of a live strain of S. aureus in preparation of a medicament for preventing and/or treating Staphylococcus aureus infection, wherein the strain lacks adenosine synthase A (AdsA) activity.

32. The use according to claim 31, wherein the live strain of S. aureus comprises a deletion of an AdsA gene encoding AdsA.

33. The use according to claim 31, wherein the live strain of S. aureus comprises a mutation in an AdsA gene encoding AdsA.

34. The use according to claim 33, wherein the mutation in the AdsA gene results in a deletion of a portion of AdsA responsible for adenosine production.

35. The use according to claim 31, wherein the AdsA activity is responsible for attenuation of NLRP-3 mediated IL-1β production in an inflammatory cell via the adenosine/A2AR pathway during Staphylococcus aureus infection.

36. The use according to claim 31, wherein the strain is derived from Staphylococcus aureus USA300, Newman, or ATCC29213.

37. The use according to claim 31, wherein the Staphylococcus aureus infection is a skin infection, soft-tissue infection, or invasive disease.

38. The use according to claim 37, wherein the invasive disease is bloodstream infection, endocarditis or sepsis.

39. The use according to claim 31, wherein the Staphylococcus aureus infection is methicillin-resistant S. aureus (MRSA) infection or MSSA infection.

40. The use according to claim 31, wherein the live strain of S. aureus is administered intramuscularly, intraperitoneally, subcutaneously, orally or intranasally, preferably, the live strain is not administered intravenously.

41. A kit for immunization against S. aureus infection, comprising a container containing the vaccine of any one of claims 1-10 or the live strain of S. aureus of any one of claims 11-20.
